(19) United States
(12) Patent Application Publication
(10) Pub. No.: US 2026/0315463 A1
(43) Pub. Date: Oct. 15, 2026

(54) METHODS FOR TREATING CONSTIPATION ASSOCIATED WITH CYSTIC FIBROSIS

(71) Applicant: Mirum Pharmaceuticals, Inc., Foster City, CA (US)

(72) Inventor: Jaya Bharati Punati, Los Angeles, CA (US)

(73) Assignee: Mirum Pharmaceuticals, Inc., Foster City, CA (US)

(21) Appl. No.: 19/645,589

(22) Filed: Apr. 13, 2026

Related U.S. Application Data
(60) Provisional application No. 63/788,360, filed on Apr. 14, 2025.
(60) Provisional application No. 63/931,009, filed on Dec. 4, 2025.

(51) Int. Cl. A61K 31/4995; A61P 1/10

(52) U.S. Cl. CPC A61K 31/4995; A61P 1/10

(57) ABSTRACT

Provided herein are methods for treating constipation associated with cystic fibrosis in a subject. The method includes administering maralixibat or a pharmaceutically acceptable salt thereof to the subject. More specifically, the present invention relates to methods for treating chronic constipation in a subject having cystic fibrosis wherein the method comprises administering an effective amount of maralixibat or a pharmaceutically acceptable salt thereof to the subject in need thereof.

### CROSS REFERENCE TO RELATED APPLICATIONS

This application claims the benefit under 35 U.S.C. § 119 (e) of U.S. Provisional Application Nos. 63/788,360, filed Apr. 14, 2025, and 63/931,009, filed Dec. 4, 2025, which are incorporated by reference in their entireties for all purposes.

### FIELD OF THE INVENTION

The present invention relates generally to methods for treating constipation associated with cystic fibrosis in a subject. More specifically, the present invention relates to methods for treating chronic constipation in a subject having cystic fibrosis wherein the method comprises administering an effective amount of maralixibat or a pharmaceutically acceptable salt thereof to the subject in need thereof.

### BACKGROUND OF THE INVENTION

Chronic constipation is a common feature cystic fibrosis (CF), affecting up to 57% of children in a recent meta-analysis (1). Symptoms can be wide-ranging from chronic abdominal pain to life-threatening gastrointestinal obstruction, such as in distal intestinal obstruction syndrome (DIOS). This is thought to result from inhibition of gastrointestinal (GI) luminal cell secretory activity due to ineffective chloride channel function due to CF mutations. The loss of these endogenous secretions results in thicker, harder-to-pass intestinal and colonic contents.

Current treatments are not always effective in relieving constipation and have practical drawbacks. Alternative hygroscopic medications (fiber-based stool softeners) require a large volume of medication that is difficult to administer in a young child. In addition, children with CF have high metabolic needs requiring high caloric intake. Large volume stool softeners displace the much-needed caloric intake and end up being counterproductive. Even stimulant laxatives are not a good option in these patients as they are ineffective when used alone due again to decreased luminal secretions.

Thus, there exists an unmet need for treatment of constipation associated with cystic fibrosis.

### SUMMARY OF THE INVENTION

Various non-limiting aspects and embodiments of the invention are described below.

In one aspect, the present disclosure provides a method for treating constipation associated with cystic fibrosis in a subject in need thereof comprising administering to the subject an effective amount of maralixibat, or a pharmaceutically acceptable salt thereof.

In one embodiment, the constipation is a chronic constipation. In one embodiment, the constipation is constipation predominant irritable bowel syndrome (IBS-C), chronic idiopathic constipation (CIC), constipation due to distal intestinal obstruction syndrome (DIOS), and/or functional constipation.

In one embodiment, the constipation is determined by analyzing stool consistency. In one embodiment, the stool consistency is measured by Bristol Stool Scale (BSS). In one embodiment, the BSS score is about grade 3 or less prior to the administration of maralixibat, or a pharmaceutically acceptable salt thereof. In one embodiment, the BSS score is about grade 3 or less for at least one week prior to the administration of maralixibat, or a pharmaceutically acceptable salt thereof.

In one embodiment, the subject is administered a constipation medication prior to the administration of maralixibat, or a pharmaceutically acceptable salt thereof. In one embodiment, the constipation medication is selected from a laxative, a stool softener, a hygroscopic medication, a secretagogue, a prokinetic, a peripherally acting mu-opioid receptor agonist (PAMORA), lactulose, sorbitol, macrogol 3350, sodium phosphate, glycerol suppository, and combination thereof. In one embodiment, the laxative is selected from bisacodyl, glycerin, magnesium hydroxide, polyethylene glycol, psyllium, senna, sodium picosulfate, docusate, and combination thereof. In one embodiment, the laxative is polyethylene glycol 3350. In one embodiment, the hygroscopic medication is a fiber-based stool softener.

In one embodiment, the subject is concurrently administered the constipation medication and maralixibat, or a pharmaceutically acceptable salt thereof. In one embodiment, the administration of the constipation medication is discontinued prior to the administration of maralixibat, or a pharmaceutically acceptable salt thereof. In one embodiment, the subject is administered maralixibat, or a pharmaceutically acceptable salt thereof, after at least four weeks from the start of the administration of a constipation medication.

In one embodiment, the maralixibat or pharmaceutically acceptable salt thereof is administered in an amount from about 100 μg/kg/day to about 1200 μg/kg/day. In one embodiment, the maralixibat or pharmaceutically acceptable salt thereof is administered in an amount from about 150 μg/kg/day to about 900 μg/kg/day. In one embodiment, the maralixibat or pharmaceutically acceptable salt thereof is administered in an amount of about 190 μg/kg/day to about 800 μg/kg/day. In one embodiment, the maralixibat or pharmaceutically acceptable salt thereof is administered in an amount of about 190 μg/kg/day or about 380 μg/kg/day. In one embodiment, the maralixibat or pharmaceutically acceptable salt thereof is administered in an amount of about 190 μg/kg/day for at least one week. In one embodiment, the maralixibat or pharmaceutically acceptable salt thereof is administered in an amount of about 380 μg/kg/day after one week of administration in an amount of about 190 μg/kg/day. In one embodiment, the maralixibat or pharmaceutically acceptable salt thereof is administered in an amount of about 380 μg/kg/day for at least one week after one week of administration in an amount of about 190 μg/kg/day. In one embodiment, the maralixibat or pharmaceutically acceptable salt thereof is administered in an amount of about 380 μg/kg/day for at least two weeks after one week of administration in an amount of about 190 μg/kg/day.

In one embodiment, the pharmaceutically acceptable salt of maralixibat is maralixibat chloride, maralixibat bromide, maralixibat acetate, or maralixibat mesylate. In one embodiment, the pharmaceutically acceptable salt of maralixibat is maralixibat chloride.

In one embodiment, the subject is a pediatric subject. In one embodiment, the subject is about 1 year old to about 18 years old.

In one embodiment, the maralixibat or pharmaceutically acceptable salt thereof is administered once daily (QD).

In one embodiment, the administration of maralixibat or pharmaceutically acceptable salt thereof increases a BSS score of the subject relative to baseline. In one embodiment, the administration of maralixibat or pharmaceutically acceptable salt thereof results in a BSS score of about grade 4 or higher.

In one embodiment, the administration of maralixibat or pharmaceutically acceptable salt thereof results in improved ease of stooling. In one embodiment, the administration of maralixibat or pharmaceutically acceptable salt thereof results in softer stool consistency.

In one embodiment, the cystic fibrosis is determined via genetic testing or sweat chloride testing.

In one embodiment, the subject does not have (i) uncontrolled fat-soluble vitamin deficiency, (ii) abnormal liver function, (iii) changes to conventional constipation medication within four weeks prior to the administration of maralixibat, or a pharmaceutically acceptable salt thereof, and/or (iv) an allergy or sensitivity to any medication involved in the treatment prior to maralixibat administration.

In one embodiment, the maralixibat or pharmaceutically acceptable salt thereof is administered before a meal. In one embodiment, the maralixibat or pharmaceutically acceptable salt thereof is administered about 30 minutes before a meal.

In one embodiment, the maralixibat is administered orally.

In one embodiment, the maralixibat is administered in the form of a pharmaceutical composition comprising maralixibat, or a pharmaceutically acceptable salt thereof, an antioxidant, and a preservative.

In one embodiment, the pharmaceutical composition is a liquid composition for oral administration. In one embodiment, the liquid composition is an aqueous solution. In one embodiment, the maralixibat is present in an amount of about 1 mg/mL to about 100 mg/ml of the composition. In one embodiment, the maralixibat is present in an amount of about 1 mg/mL to about 50 mg/mL of the composition. In one embodiment, the maralixibat is present in an amount of about 1 mg/mL to about 10 mg/mL of the composition. In one embodiment, the maralixibat is present in an amount of about 9.5 mg/mL of the composition.

In one embodiment, the pharmaceutical composition is a solid composition for oral administration.

These and other aspects of the present invention will become apparent to those skilled in the art after a reading of the following detailed description of the invention, including the appended claims.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Bile acids/salts play a critical role in activating digestive enzymes and solubilizing fats and fat-soluble vitamins and are involved in liver, biliary, and intestinal disease. Bile acids are synthesized in the liver by a multistep, multiorganelle pathway. Hydroxyl groups are added to specific sites on the steroid structure, the double bond of the cholesterol B ring is reduced, and the hydrocarbon chain is shortened by three carbon atoms resulting in a carboxyl group at the end of the chain. The most common bile acids are cholic acid and chenodeoxycholic acid (the “primary bile acids”). Before exiting the hepatocytes and forming bile, the bile acids are conjugated to either glycine (to produce glycocholic acid or glycochenodeoxycholic acid) or taurine (to produce taurocholic acid or taurochenodeoxycholic acid). The conjugated bile acids are called bile salts and their amphipathic nature makes them more efficient detergents than bile acids. Bile salts, not bile acids, are found in bile.

Bile salts are excreted by the hepatocytes into the canaliculi to form bile. The canaliculi drain into the right and left hepatic ducts and the bile flows to the gallbladder. Bile is released from the gallbladder and travels to the duodenum, where it contributes to the metabolism and degradation of fat. The bile salts are reabsorbed in the terminal ileum and transported back to the liver via the portal vein. Bile salts often undergo multiple enterohepatic circulations before being excreted via feces. A small percentage of bile salts may be reabsorbed in the proximal intestine by either passive or carrier-mediated transport processes. Most bile salts are reclaimed in the distal ileum by a sodium-dependent apically located bile acid transporter referred to as apical sodium-dependent bile acid transporter (ASBT). At the basolateral surface of the enterocyte, a truncated version of ASBT is involved in vectoral transfer of bile acids/salts into the portal circulation. Completion of the enterohepatic circulation occurs at the basolateral surface of the hepatocyte by a transport process that is primarily mediated by a sodium-dependent bile acid transporter. Intestinal bile ac-id transport plays a key role in the enterohepatic circulation of bile salts. Molecular analysis of this process has recently led to important advances in understanding of the biology, physiology and pathophysiology of intestinal bile acid transport.

Maralixibat is known as Livmarli®, SHP625, LUM001, lopixibat, or 1-[[4-[[4-[(4R,5R)-3,3-dibutyl-7-(dimethylamino)-2,3,4,5-tetrahydro-4-hydroxy-1,1-dioxido-1-benzothiepin-5-yl]phenoxy]methyl]phenyl]methyl]-4-aza-1-azoniabicyclo[2.2.2]octane. The structure of maralixibat in the free base form is shown below:

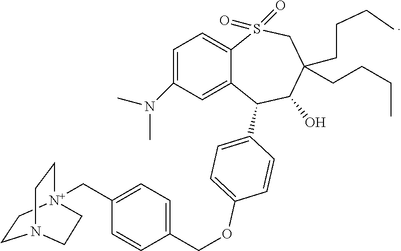 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O]

The structure of maralixibat chloride is shown below:

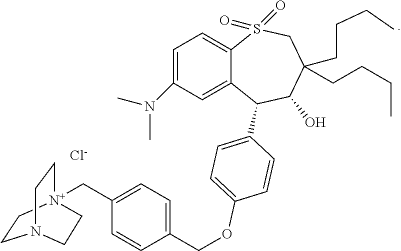 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present embodiments include pharmaceutically acceptable salt of the compounds described herein. The present embodiments also include quaternary ammonium salts of the compounds described herein, where the compounds have one or more tertiary amine moieties. In one embodiment, the pharmaceutically acceptable salt of maralixibat is maralixibat chloride.

The IBAT is ideally suited for pharmacological modulation of bile acid transport by a compound that can be restricted to the lumen of the gut, i.e., one that is not required to have systemic exposure for activity. Maralixibat was designed to be minimally absorbed due to its large molecular weight (~710 Da) and the presence of a positively charged quaternary nitrogen atom, therefore maximizing the local exposure of the molecule to its target and minimizing unnecessary systemic exposure.

### Bile Acid

Bile contains water, electrolytes and a numerous organic molecules including bile acids, cholesterol, phospholipids and bilirubin. Bile is secreted from the liver and stored in the gall bladder, and upon gall bladder contraction, due to ingestion of a fatty meal, bile passes through the bile duct into the intestine. Bile acids/salts are critical for digestion and absorption of fats and fat-soluble vitamins in the small intestine. Adult humans produce 400 to 800 mL of bile daily. The secretion of bile can be considered to occur in two stages. Initially, hepatocytes secrete bile into canaliculi, from which it flows into bile ducts and this hepatic bile contains large quantities of bile acids, cholesterol and other organic molecules. Then, as bile flows through the bile ducts, it is modified by addition of a watery, bicarbonate-rich secretion from ductal epithelial cells. Bile is concentrated, typically five-fold, during storage in the gall bladder.

The flow of bile is lowest during fasting, and a majority of that is diverted into the gallbladder for concentration. When chyme from an ingested meal enters the small intestine, acid and partially digested fats and proteins stimulate secretion of cholecystokinin and secretin, both of which are important for secretion and flow of bile. Cholecystokinin (cholecysto=gallbladder and kinin=movement) is a hormone which stimulates contractions of the gallbladder and common bile duct, resulting in delivery of bile into the gut. The most potent stimulus for release of cholecystokinin is the presence of fat in the duodenum. Secretin is a hormone secreted in response to acid in the duodenum, and it simulates biliary duct cells to secrete bicarbonate and water, which expands the volume of bile and increases its flow out into the intestine.

Bile acids/salts are derivatives of cholesterol. Cholesterol, ingested as part of the diet or derived from hepatic synthesis, are converted into bile acids/salts in the hepatocyte. Examples of such bile acids/salts include cholic and chenodeoxycholic acids, which are then conjugated to an amino acid (such as glycine or taurine) to yield the conjugated form that is actively secreted into cannaliculi. The most abundant of the bile salts in humans are cholate and deoxycholate, and they are normally conjugated with either glycine or taurine to give glycocholate or taurocholate respectively.

Free cholesterol is virtually insoluble in aqueous solutions, however in bile it is made soluble by the presence of bile acids/salts and lipids. Hepatic synthesis of bile acids/salts accounts for the majority of cholesterol breakdown in the body. In humans, roughly 500 mg of cholesterol are converted to bile acids/salts and eliminated in bile every day. Therefore, secretion into bile is a major route for elimination of cholesterol. Large amounts of bile acids/salts are secreted into the intestine every day, but only relatively small quantities are lost from the body. This is because approximately 95% of the bile acids/salts delivered to the duodenum are absorbed back into blood within the ileum, by a process is known as “Enterohepatic Recirculation”.

Venous blood from the ileum goes straight into the portal vein, and hence through the sinusoids of the liver. Hepatocytes extract bile acids/salts very efficiently from sinusoidal blood, and little escapes the healthy liver into systemic circulation. Bile acids/salts are then transported across the hepatocytes to be resecreted into canaliculi. The net effect of this enterohepatic recirculation is that each bile salt molecule is reused about 20 times, often two or three times during a single digestive phase. Bile biosynthesis represents the major metabolic fate of cholesterol, accounting for more than half of the approximate 800 mg/day of cholesterol that an average adult uses up in metabolic processes. In comparison, steroid hormone biosynthesis consumes only about 50 mg of cholesterol per day. Much more that 400 mg of bile salts is required and secreted into the intestine per day, and this is achieved by re-cycling the bile salts. Most of the bile salts secreted into the upper region of the small intestine are absorbed along with the dietary lipids that they emulsified at the lower end of the small intestine. They are separated from the dietary lipid and returned to the liver for re-use. Recycling thus enables 20-30 g of bile salts to be secreted into the small intestine each day.

Bile acids/salts are amphipathic, with the cholesterol-derived portion containing both hydrophobic (lipid soluble) and polar (hydrophilic) moieties while the amino acid conjugate is generally polar and hydrophilic. This amphipathic nature enables bile acids/salts to carry out two important functions: emulsification of lipid aggregates and solubilization and transport of lipids in an aqueous environment. Bile acids/salts have detergent action on particles of dietary fat which causes fat globules to break down or to be emulsified. Emulsification is important since it greatly increases the surface area of fat available for digestion by lipases which cannot access the inside of lipid droplets. Furthermore, bile acids/salts are lipid carriers and are able to solubilize many lipids by forming micelles and are critical for transport and absorption of the fat-soluble vitamins.

The term “non-systemic” or “minimally absorbed,” as used herein, refers to low systemic bioavailability and/or absorption of an administered compound. In some embodiments a non-systemic compound is a compound that is substantially not absorbed systemically. In some embodiments, ASBTI compositions described herein deliver the ASBTI to the distal ileum, colon, and/or rectum and not systemically (e.g., a substantial portion of the ASBTI is not systemically absorbed. In some embodiments, the systemic absorption of a non-systemic compound is <0.1%, <0.3%, <0.5%, <0.6%, <0.7%, <0.8%, <0.9%, <1%, <1.5%, <2%, <3%, or <5% of the administered dose (wt. % or mol %). In some embodiments, the systemic absorption of a non-systemic compound is <10% of the administered dose. In some embodiments, the systemic absorption of a non-systemic compound is <15% of the administered dose. In some embodiments, the systemic absorption of a non-systemic compound is <25% of the administered dose. In an alternative approach, a non-systemic ASBTI (e.g., maralixibat) is a compound that has lower systemic bioavailability relative to the systemic bioavailability of a systemic ASBTI. In some embodiments, the bioavailability of a non-systemic ASBTI described herein (e.g., maralixibat) is <30%, <40%, <50%, <60%, or <70% of the bioavailability of a systemic ASBTI.

In another alternative approach, compositions described herein are formulated to deliver <10% of the administered dose of the ASBTI systemically. In some embodiments, the compositions described herein are formulated to deliver <20% of the administered dose of the ASBTI systemically. In some embodiments, the compositions described herein are formulated to deliver <30% of the administered dose of the ASBTI systemically. In some embodiments, the compositions described herein are formulated to deliver <40% of the administered dose of the ASBTI systemically. In some embodiments, the compositions described herein are formulated to deliver <50% of the administered dose of the ASBTI systemically. In some embodiments, the compositions described herein are formulated to deliver <60% of the administered dose of the ASBTI systemically. In some embodiments, the compositions described herein are formulated to deliver <70% of the administered dose of the ASBTI systemically. In some embodiments, systemic absorption is determined in any suitable manner, including the total circulating amount, the amount cleared after administration, or the like.

Bile acids/salts play a critical role in activating digestive enzymes and solubilizing fats and fat-soluble vitamins and are involved in liver, biliary, and intestinal disease. Bile acids are synthesized in the liver by a multistep, multiorganelle pathway. Hydroxyl groups are added to specific sites on the steroid structure, the double bond of the cholesterol B ring is reduced, and the hydrocarbon chain is shortened by three carbon atoms resulting in a carboxyl group at the end of the chain. The most common bile acids are cholic acid and chenodeoxycholic acid (the “primary bile acids”). Before exiting the hepatocytes and forming bile, the bile acids are conjugated to either glycine (to produce glycocholic acid or glycochenodeoxycholic acid) or taurine (to produce taurocholic acid or taurochenodeoxycholic acid). The conjugated bile acids are called bile salts and their amphipathic nature makes them more efficient detergents than bile acids. Bile salts, not bile acids, are found in bile.

Bile salts are excreted by the hepatocytes into the canaliculi to form bile. The canaliculi drain into the right and left hepatic ducts and the bile flows to the gallbladder. Bile is released from the gallbladder and travels to the duodenum, where it contributes to the metabolism and degradation of fat. The bile salts are reabsorbed in the terminal ileum and transported back to the liver via the portal vein. Bile salts often undergo multiple enterohepatic circulations before being excreted via feces. A small percentage of bile salts may be reabsorbed in the proximal intestine by either passive or carrier-mediated transport processes. Most bile salts are reclaimed in the distal ileum by a sodium-dependent apically located bile acid transporter referred to as apical sodium-dependent bile acid transporter (ASBT). At the basolateral surface of the enterocyte, a truncated version of ASBT is involved in vectoral transfer of bile acids/salts into the portal circulation. Completion of the enterohepatic circulation occurs at the basolateral surface of the hepatocyte by a transport process that is primarily mediated by a sodium-dependent bile acid transporter. Intestinal bile acid transport plays a key role in the enterohepatic circulation of bile salts. Molecular analysis of this process has recently led to important advances in understanding of the biology, physiology, and pathophysiology of intestinal bile acid transport.

Within the intestinal lumen, bile acid concentrations vary, with the bulk of the reuptake occurring in the distal intestine. Described herein are certain compositions and methods that control bile acid concentrations in the intestinal lumen, thereby controlling the hepatocellular damage caused by bile acid accumulation in the liver.

Constipation Associated with Cystic Fibrosis

Constipation is a common gastrointestinal manifestation in individuals with Cystic Fibrosis (CF). This condition is characterized by a reduced frequency of bowel movements and an increase in stool consistency. Additionally, it is often accompanied by abdominal pain and distension. One of the primary etiological factors contributing to constipation in CF patients is an altered composition of intestinal fluids, which is a result of the defective expression of the Cystic Fibrosis Transmembrane Conductance Regulator (CFTR) protein in the gut. The diagnosis of constipation relies heavily on a thorough medical history and a detailed physical examination. These are key elements in identifying the condition accurately. Given that the symptoms and underlying pathophysiology of constipation in CF patients differ significantly from those in individuals without CF, the Rome criteria, which are typically used to diagnose functional gastrointestinal disorders, may not be applicable. Consequently, an ESPGHAN (European Society for Paediatric Gastroenterology Hepatology and Nutrition) working group convened to establish specific diagnostic criteria for constipation in CF patients. The resulting definition includes the presence of abdominal pain, abdominal distension, a reduced frequency of bowel movements, or increased stool consistency, as observed over the past few weeks to months. The pathophysiology of constipation in CF is multifactorial. It involves a slower transit time through the small intestine and a different composition of intestinal contents, which includes more viscous mucus and altered pH levels. Additionally, low-grade intestinal inflammation, as indicated by elevated calprotectin levels, and an abnormal microbiome composition contribute to the condition. Other contributing factors may include variations in small bowel water content, prolonged oral-caecal transit time, and changes in colon fasting volume.

Constipation associated with cystic fibrosis (CF) is often linked to the thick mucus produced which can obstruct the intestines, leading to slower bowel movements and harder stools. A Bristol Stool Scale (BSS) score indicating a harder stool consistency (lower numbers on the scale) is generally be observed in a CF patient experiencing constipation.

### ASBT Inhibitors

In various embodiments of methods of the present invention, an ASBT inhibitor is administered to a subject. In some embodiments, the ASBTI inhibitor is maralixibat, or a pharmaceutically acceptable salt thereof. In some embodiments, the ASBTI inhibitor is maralixibat chloride. In certain embodiments, the ASBTIs are systemically absorbed. In certain embodiments, the ASBTIs are not systemically absorbed. In one embodiment, maralixibat is a non-systemically absorbed ASBTI. In some embodiments, the ASBTI used in the methods or compositions of the present invention is maralixibat, or a pharmaceutically acceptable salt thereof.

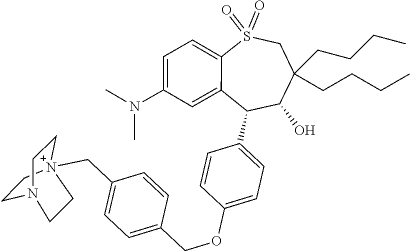 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O]

In one embodiment, the ASBTI used in the methods or compositions of the present invention is (maralixibat chloride, LUM-001, SHP625, lopixibat chloride).

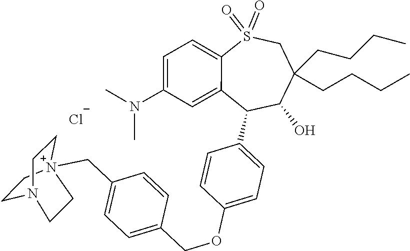 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

In one embodiment, the ASBTI used in the methods or compositions of the present invention is maralixibat bromide.

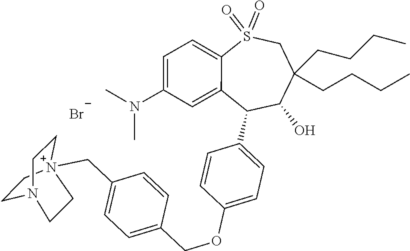 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Br-]]

In one embodiment, the ASBTI used in the methods or compositions of the present invention is maralixibat acetate.

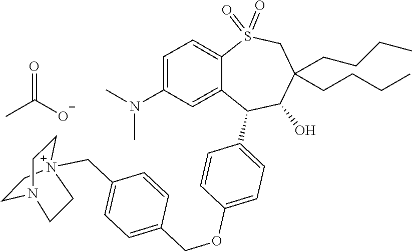

In one embodiment, the ASBTI used in the methods or compositions of the present invention is maralixibat mesylate.

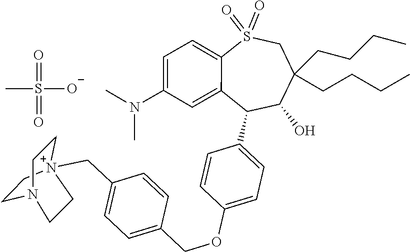

Maralixibat is a non-systemic ileal bile acid transport inhibitor (IBATi) that acts by interrupting bile acid reabsorption in the ileum thus interrupting the normal enterohepatic circulation. This interruption results in a larger volume of bile acids reaching the colon and being excreted in stool. Bile acids are known to decrease bowel transit time, increase mucosal permeability and secretions, and alter gut microbiota resulting in diarrhea. (2-7)

Multiple adult studies showed a reduction in colonic transit time and an increase in frequency of bowel movements in adult patients with constipation predominant irritable bowel syndrome (IBS-C), chronic idiopathic constipation (CIC) or Functional constipation (8-11). Chey et al. showed a similar response in 190 adult patients with CIC over a period of 8 weeks (10). There are no pediatric studies thus far assessing similar improvement in children.

In children, maralixibat is an oral liquid solution (concentration 9.5 mg/mL) approved and indicated for the treatment of cholestatic pruritus in Alagille Syndrome (ALGS) and Progressive Familial Intrahepatic Cholestasis (PFIC) for patients age 1 year or older. The approved dosing regimen is 190 μg/kg orally once daily with titration to 380 μg/kg orally once daily after one week, as tolerated, based on maralixibat free base (12, see Section 2.1 Dosing in package insert for detailed dosing instructions). Diarrhea is listed as the most common adverse reaction in ALGS patients on maralixibat (12, 13), which is attributed to increased bile acid delivery to the colon.

Methods for Treating Constipation Associated with Cystic Fibrosis

Provided herein is a method for treating constipation in a subject having a cystic fibrosis. The method comprises administering to a subject in need of treatment an Apical Sodium-dependent Bile Acid Transporter Inhibitor (ASBTI). In one embodiment, the ASBTI is maralixibat, maralixibat chloride, or an alternative pharmaceutically acceptable salt thereof. In one embodiment, the ASBTI is administered in an amount of from about 100 μg/kg/day to about 1200 μg/kg/day.

In various embodiments, the constipation is a chronic constipation. In one embodiment, the chronic constipation is constipation predominant irritable bowel syndrome (IBS-C), constipation due to distal intestinal obstruction syndrome (DIOS), chronic idiopathic constipation (CIC), and/or functional constipation.

In certain embodiments, the constipation is determined by analyzing stool consistency. A non-limiting example of analyzing stool consistency is a measurement of Bristol Stool Scale (BSS). The BSS score of about grade 3 or less can be considered as an indication of constipation that needs to be treated. In some embodiments, the subject has a BSS score of about grade 3 or less prior to the administration of maralixibat, a pharmaceutically acceptable salt thereof, or a composition described herein. The BSS score of about grade 3 or less can also be considered as a baseline to determine the effect of the methods or compositions of the present disclosure.

In some embodiments, the treatment results in a BSS score of about grade 4 or higher. In some embodiments, the treatment results in improved ease of stooling. The terms ease of stooling and ease of defecation are used interchangeably. In some embodiments, the treatment results in softer stool consistency.

In some embodiments, the subject is a pediatric subject. In various embodiments, the patient is a pediatric patient under the age of 1 month, 2 months, 3 months, 4 months, 5 months, 6 months, 7 months, 8 months, 9 months, 10 months, 11 months, or 1, 2, 3, 4, 5, 6, 7, 8, 9, 10, 11, 12, 13, 14, 15, 16, 17, or 18 years old. In certain embodiments, the pediatric subject is a newborn, a pre-term newborn, an infant, a toddler, a preschooler, a school-age child, a pre-pubescent child, post-pubescent child, an adolescent, or a teenager under the age of eighteen. In some embodiments, the pediatric subject is a newborn, a pre-term newborn, an infant, a toddler, a preschooler, or a school-age child. In some embodiments, the pediatric subject is a newborn, a pre-term newborn, an infant, a toddler, or a preschooler. In some embodiments, the pediatric subject is a newborn, a pre-term newborn, an infant, or a toddler. In some embodiments, the pediatric subject is a newborn, a pre-term newborn, or an infant. In some embodiments, the pediatric subject is a newborn. In some embodiments, the pediatric subject is an infant. In some embodiments, the pediatric subject is a toddler. In some embodiments, the pediatric subject is at least 1 year of age.

In some embodiments, the subject is an adult. In some embodiments, the subject is over 18 years of age, or over 25 years of age, or over 50 years of age, or over 60 years of age, or over 65 years of age, or over 70 years of age, or over 75 years of age. In certain embodiments, methods of the present invention comprise non-systemic administration of a therapeutically effective amount of maralixibat or maralixibat chloride. In certain embodiments, the methods comprise contacting the gastrointestinal tract, including the distal ileum and/or the colon and/or the rectum, of an individual in need thereof with maralixibat or maralixibat chloride.

In various embodiments, methods of the present invention comprise delivering to ileum or colon of the individual a therapeutically effective amount of maralixibat or maralixibat chloride.

Administration of a compound described herein may be achieved in any suitable manner including, by way of non-limiting example, by oral, enteric, parenteral (e.g., intravenous, subcutaneous, intramuscular), intranasal, buccal, topical, rectal, or transdermal administration routes. Any compound or composition described herein may be administered in a method or formulation appropriate to treat a newborn or an infant. Any compound or composition described herein may be administered in an oral formulation (e.g., solid or liquid) to treat a newborn or an infant. Any compound or composition described herein may be administered prior to ingestion of food, with food or after ingestion of food.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein may be administered to an individual susceptible to or otherwise at risk of a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain embodiments of the methods of the present invention, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain embodiments of the methods of the present invention, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In some embodiments, doses administered include those up to the maximum tolerable dose by a newborn or an infant.

In various embodiments of the methods of the present invention, a desired dose is conveniently presented in a single dose or in divided doses administered simultaneously (or over a short period of time) or at appropriate intervals, for example as two, three, four or more sub-doses per day. In various embodiments, a single dose of maralixibat or maralixibat chloride is administered every 6 hours, every 12 hours, every 24 hours, every 48 hours, every 72 hours, every 96 hours, every 5 days, every 6 days, or once a week. In some embodiments the total single dose of maralixibat or maralixibat chloride is in a range described below.

In various embodiments of methods of the present invention, in the case wherein the patient's status does improve, upon the doctor's discretion maralixibat or maralixibat chloride is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100% of the original dose, including, by way of example only, 10%, 15%, 20%, 25%, 30%, 35%, 40%, 45%, 50%, 55%, 60%, 65%, 70%, 75%, 80%, 85%, 90%, 95%, or 100% of the original dose. In some embodiments, the total single dose of an ASBTI is in a range described below.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

### Dosages

In various embodiments, the ASBTI is maralixibat, or a pharmaceutically acceptable salt thereof.

In various embodiments, the efficacy of ASBTI administration to the patient is monitored by measuring a BSS score.

In various embodiments, the safety of ASBTI administration to the patient is monitored by identifying the occurrence of one or more side effects. The one or more side effects may include, but not limited to, diarrhea, abdominal pain, vomiting, liver test abnormalities, fat-soluble vitamin deficiency, nausea, gastrointestinal bleeding, and bone fractures.

In some embodiments, the ASBTI is administered at a dose of about or at least about 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 190 μg/kg, 200 μg/kg, 240 μg/kg, 250 μg/kg, 280 μg/kg, 300 μg/kg, 380 μg/kg, 400 μg/kg, 500 μg/kg, 560 μg/kg, 600 μg/kg, 700 μg/kg, 760 μg/kg, 800 μg/kg, 900 μg/kg, 1,000 μg/kg, 1,100 μg/kg, 1,200 μg/kg, 1,300 μg/kg, 1,400 μg/kg, 1500 μg/kg, 1,600 μg/kg, 1,700 μg/kg, 1,800 μg/kg, 1,900 μg/kg, or 2,000 μg/kg. In various embodiments, the ASBTI is administered at a dose not exceeding about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 190 μg/kg, 200 μg/kg, 240 μg/kg, 250 μg/kg, 280 μg/kg, 300 μg/kg, 380 μg/kg, 400 μg/kg, 500 μg/kg, 560 μg/kg, 600 μg/kg, 700 μg/kg, 760 μg/kg, 800 μg/kg, 900 μg/kg, 1,000 μg/kg, 1,100 μg/kg, 1,200 μg/kg, 1,300 μg/kg, 1,400 μg/kg, 1,500 μg/kg, 1,600 μg/kg, 1,700 μg/kg, 1,800 μg/kg, 1,900 μg/kg, 2,000, or 2,100 μg/kg. In various embodiments, the ASBTI is administered at a dose of about or of at least about 0.1 mg/day, 0.2 mg/day, 0.3 mg/day, 0.4 mg/day, 0.5 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, or 1000 mg/day. In various embodiments, the ASBTI is administered at a dose of not more than about 0.1 mg/day, 0.2 mg/day, 0.3 mg/day, 0.4 mg/day, 0.5 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1,000 mg/day, or 1,100 mg/day.

In some embodiments, maralixibat is administered at a dose of from about 140 μg/kg/day to about 1400 μg/kg/day. In some embodiments, maralixibat is administered at a dose of from about 100 μg/kg/day to about 1200 μg/kg/day. In various embodiments, maralixibat is administered at a dose of about or at least about 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 190 μg/kg/day, 200 μg/kg/day, 240 μg/kg/day, 250 μg/kg/day, 280 μg/kg/day, 300 μg/kg/day, 380 μg/kg/day, 400 μg/kg/day, 500 μg/kg/day, 560 μg/kg/day, 600 μg/kg/day, 700 μg/kg/day, 760 μg/kg, 800 μg/kg/day, 900 μg/kg/day, 1,000 μg/kg/day, 1,100 μg/kg/day, 1,200 μg/kg/day, or 1,300 μg/kg/day. In various embodiments, maralixibat is administered at a dose not exceeding about 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 g/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 190 μg/kg/day, 200 μg/kg/day, 240 μg/kg/day, 280 μg/kg/day, 300 μg/kg/day, 250 μg/kg/day, 280 μg/kg/day, 300 μg/kg/day, 380 μg/kg/day, 400 μg/kg/day, 500 μg/kg/day, 560 μg/kg/day, 600 μg/kg/day, 700 μg/kg/day, 760 μg/kg, 800 μg/kg/day, 900 μg/kg/day, 1,000 μg/kg/day, 1,100 μg/kg/day, 1,200 μg/kg/day, 1,300 μg/kg/day, or 1,400 μg/kg/day. In various embodiments, maralixibat is administered at a dose of from about 0.5 μg/kg/day to about 500 μg/kg/day, from about 0.5 μg/kg/day to about 250 μg/kg/day, from about 1 μg/kg/day to about 100 μg/kg/day, from about 10 μg/kg/day to about 50 μg/kg/day, from about 10 μg/kg/day to about 100 μg/kg/day, from about 0.5 μg/kg/day to about 2000 μg/kg/day, from about 100 μg/kg/day to about 1200 μg/kg/day, from about 150 μg/kg/day to about 900 μg/kg/day, from about 180 μg/kg/day to about 800 μg/kg/day, from about 190 μg/kg/day to about 380 μg/kg/day, from about 280 μg/kg/day to about 1400 μg/kg/day, from about 420 μg/kg/day to about 1400 μg/kg/day, from about 250 to about 550 μg/kg/day, from about 560 μg/kg/day to about 1400 μg/kg/day, from 700 μg/kg/day to about 1400 μg/kg/day, from about 560 μg/kg/day to about 1200 μg/kg/day, from about 700 μg/kg/day to about 1200 μg/kg/day, from about 560 μg/kg/day to about 1000 μg/kg/day, from about 700 μg/kg/day to about 1000 μg/kg/day, from about 800 μg/kg/day to about 1000 μg/kg/day, from about 200 μg/kg/day to about 600 μg/kg/day, from about 300 μg/kg/day to about 600 μg/kg/day, from about 400 μg/kg/day to about 500 μg/kg/day, from about 400 μg/kg/day to about 600 μg/kg/day, from about 400 μg/kg/day to about 700 μg/kg/day, from about 400 μg/kg/day to about 800 μg/kg/day, from about 500 μg/kg/day to about 800 μg/kg/day, from about 500 μg/kg/day to about 900 μg/kg/day, from about 600 μg/kg/day to about 900 μg/kg/day, from about 700 μg/kg/day to about 900 μg/kg/day, from about 200 μg/kg/day to about 600 μg/kg/day, from about 800 μg/kg/day to about 900 μg/kg/day, from about 100 μg/kg/day to about 1500 μg/kg/day, from about 300 μg/kg/day to about 2,000 μg/kg/day, or from about 400 μg/kg/day to about 2000 μg/kg/day.

In some embodiments, maralixibat is administered at a dose of from about 30 μg/kg to about 1400 μg/kg per dose. In some embodiments, maralixibat is administered at a dose of from about 100 μg/kg to about 1200 μg/kg per dose. In some embodiments, maralixibat is administered at a dose of from about 150 μg/kg to about 900 μg/kg per dose. In some embodiments, maralixibat is administered at a dose of from about 180 μg/kg to about 800 μg/kg per dose. In some embodiments, maralixibat is administered at a dose of from about 190 μg/kg to about 380 μg/kg per dose. In some embodiments, maralixibat is administered at a dose of from about 0.5 μg/kg to about 2000 μg/kg per dose, from about 0.5 μg/kg to about 1500 μg/kg per dose, from about 100 μg/kg to about 700 μg/kg per dose, from about 5 μg/kg to about 100 μg/kg per dose, from about 10 μg/kg to about 500 μg/kg per dose, from about 50 μg/kg to about 1400 μg/kg per dose, from about 100 μg/kg to about 1200 μg/kg per dose, from about 150 μg/kg to about 900 μg/kg per dose, from about 180 μg/kg to about 800 μg/kg per dose, from about 300 μg/kg to about 2,000 μg/kg per dose, from about 60 μg/kg to about 1200 μg/kg per dose, from about 70 μg/kg to about 1000 μg/kg per dose, from about 70 μg/kg to about 700 μg/kg per dose, from 80 μg/kg to about 1000 μg/kg per dose, from 80 μg/kg to about 800 μg/kg per dose, from 100 μg/kg to about 800 μg/kg per dose, from 100 μg/kg to about 600 μg/kg per dose, from 150 μg/kg to about 700 μg/kg per dose, from 150 μg/kg to about 500 μg/kg per dose, from 200 μg/kg to about 400 μg/kg per dose, from 200 μg/kg to about 300 μg/kg per dose, from 300 μg/kg to about 400 μg/kg per dose, from 400 μg/kg to about 800 μg/kg per dose, from 500 μg/kg to about 700 μg/kg per dose, or from 5500 μg/kg to about 650 μg/kg per dose.

In some embodiments, maralixibat is administered at a dose of from about 0.05 mg/day to about 550 mg/day. In some embodiments, maralixibat is administered at a dose of from about 0.5 mg/day to about 550 mg/day. In various embodiments, maralixibat is administered at a dose of from about 0.05 mg/day to about 10 mg/day, from about 0.05 mg/day to about 1 mg/day, from about 0.1 mg/day to about 10 mg/day, from about 0.1 mg/day to about 1 mg/day, from about 0.05 mg/day to about 0.5 mg/day, from about 0.1 mg/day to about 0.5 mg/day, from about 1 mg/day to about 500 mg/day, from about 1 mg/day to about 300 mg/day, from about 1 mg/day to about 200 mg/day, from about 2 mg/day to about 300 mg/day, from about 2 mg/day to about 200 mg/day, from about 4 mg/day to about 300 mg/day, from about 4 mg/day to about 200 mg/day, from about 4 mg/day to about 150 mg/day, from about 5 mg/day to about 150 mg/day, from about 5 mg/day to about 100 mg/day, from about 5 mg/day to about 80 mg/day, from about 5 mg/day to about 50 mg/day, from about 5 mg/day to about 40 mg/day, from about 5 mg/day to about 30 mg/day, from about 5 mg/day to about 20 mg/day, from about 5 mg/day to about 15 mg/day, from about 10 mg/day to about 100 mg/day, from about 10 mg/day to about 80 mg/day, from about 10 mg/day to about 50 mg/day, from about 10 mg/day to about 40 mg/day, from about 10 mg/day to about 20 mg/day, from about 20 mg/day to about 100 mg/day, from about 20 mg/day to about 80 mg/day, from about 20 mg/day to about 50 mg/day, or from about 20 mg/day to about 40 mg/day, from about 20 mg/day to about 30 mg/day, from about 30 mg/day to about 80 mg/day, from about 40 mg/day to about 70 mg/day, or from about 50 mg/day to about 60 mg/day.

In some embodiments, maralixibat is administered twice daily (BID) in an amount of about 50 μg/kg to about 250 μg/kg per dose. In some embodiments, maralixibat is administered twice daily (BID) in an amount of about 150 μg/kg to about 600 μg/kg per dose. In some embodiments, maralixibat is administered in an amount of about 100 μg/kg/day to about 500 μg/kg/day. In some embodiments, maralixibat is administered in an amount of about 150 μg/kg/day to about 400 μg/kg/day. In some embodiments, maralixibat is administered in an amount of about 280 μg/kg/day to about 1400 μg/kg/day. In some embodiments, maralixibat is administered in an amount of about 400 μg/kg/day to about 800 μg/kg/day. In some embodiments, maralixibat is administered in an amount of about 10 mg/day to about 100 mg/day. In some embodiments, maralixibat is administered in an amount of from about 15 mg/day to about 60 mg/day. In some embodiments, maralixibat is administered in an amount of from about 560 μg/kg/day to about 1,200 μg/kg/day. In some embodiments, maralixibat is administered in an amount of from about 700 μg/kg/day to about 1,400 μg/kg/day. In some embodiments, maralixibat is administered in an amount of from about 400 μg/kg/day to about 800 μg/kg/day. In some embodiments, maralixibat is administered in an amount of from about 700 μg/kg/day to about 900 μg/kg/day. In some embodiments, maralixibat is administered in an amount of from about 560 μg/kg/day to about 1400 μg/kg/day. In some embodiments, maralixibat is administered in an amount from 700 μg/kg/day to about 1400 μg/kg/day. In some embodiments, maralixibat is administered in an amount of from about 200 μg/kg/day to about 600 μg/kg/day. In some embodiments, maralixibat is administered in an amount of from about 400 μg/kg/day to about 600 μg/kg/day. In some embodiments, maralixibat is administered in an amount of from about 1100 μg/kg/day to about 1200 μg/kg/day.

In various embodiments, the dose of maralixibat is a first dose level. In various embodiments, the dose of maralixibat is a second dose level. In various embodiments, the dose of maralixibat is a third dose level. In various embodiments, the dose of maralixibat is a fourth dose level. In some embodiments, the second dose level is greater than the first dose level. In some embodiments, the second dose level is about or at least about 1.5, 2, 3, 4, 5, 6, 7, 8, 9, 10, 15, 20, 30, 40, 50, 60, 70, 80, 90 or 100 times or fold greater than the first dose level. In some embodiments, the second dose level is not in excess of about 1.5, 2, 3, 4, 5, 6, 7, 8, 9, 10, 15, 20, 30, 40, 50, 60, 70, 80, 90, 100, or 150 times or fold greater than the first dose level. In some embodiments, the third dose level is greater than the second dose level. In some embodiments, the third dose level is about or at least about 1.5, 2, 3, 4, 5, 6, 7, 8, 9, 10, 15, 20, 30, 40, 50, 60, 70, 80, 90 or 100 times or fold greater than the second dose level. In some embodiments, the third dose level is not in excess of about 1.5, 2, 3, 4, 5, 6, 7, 8, 9, 10, 15, 20, 30, 40, 50, 60, 70, 80, 90, 100, or 150 times or fold greater than the second dose level. In some embodiments, the fourth dose level is greater than the third dose level. In some embodiments, the fourth dose level is about or at least about 1.5, 2, 3, 4, 5, 6, 7, 8, 9, 10, 15, 20, 30, 40, 50, 60, 70, 80, 90 or 100 times or fold greater than the third dose level. In some embodiments, the fourth dose level is not in excess of about 1.5, 2, 3, 4, 5, 6, 7, 8, 9, 10, 15, 20, 30, 40, 50, 60, 70, 80, 90, 100, or 150 times or fold greater than the third dose level.

In some embodiments, maralixibat is administered once daily (QD) in an amount of about 100 μg/kg to about 1200 μg/kg. In some embodiments, maralixibat is administered once daily in an amount of about 150 μg/kg to about 900 μg/kg. In some embodiments, maralixibat is administered once daily in an amount of about 180 μg/kg to about 800 μg/kg. In some embodiments, maralixibat is administered once daily in an amount of about 190 μg/kg to about 380 μg/kg. In some embodiments, maralixibat is administered once daily in an amount of about 190 μg/kg. In some embodiments, maralixibat is administered once daily in an amount of about 380 μg/kg.

In various embodiments, maralixibat is administered once daily (QD) at one of the above doses or within one of the above dose ranges. In various embodiments, maralixibat is administered twice daily (BID) at one of the above doses or within one of the above dose ranges. In various embodiments, an ASBTI dose is administered daily, every other day, twice a week, or once a week.

In various embodiments, maralixibat is administered regularly for a period of about or of at least about 1, 2, 3, 4, 5, 6, 7, 8, 9, 10, 15, 20, 30, 40, 48, 50, 75, 100, 150, 200, 250, 300, 350, 400, 450, 500, 600, 700, or 800 weeks. In various embodiments, maralixibat is administered for not more than about 1, 2, 3, 4, 5, 6, 7, 8, 9, 10, 15, 20, 30, 40, 48, 50, 75, 100, 150, 200, 250, 300, 350, 400, 450, 500, 600, 700, 800, or 1000 weeks. In various embodiments, maralixibat is administered regularly for a period of about or of at least about 0.5, 1, 1.5, 2, 3, 4, 5, 6, 7, 8, 9, or 10 years. In various embodiments, maralixibat is administered regularly for a period not in excess of about 0.5, 1, 1.5, 2, 3, 4, 5, 6, 7, 8, 9, 10, or 15 years.

### Combination Therapy

In various embodiments of the above methods of the invention, maralixibat or a pharmaceutically acceptable salt thereof is administered in combination with a constipation medication. In some embodiments, a constipation medication is administered prior to the administration of maralixibat or a pharmaceutically acceptable salt thereof. In some embodiments, the constipation medication is administered prior to the administration of maralixibat or a pharmaceutically acceptable salt thereof and is discontinued upon the start of the administration of maralixibat or a pharmaceutically acceptable salt thereof. In some embodiments, a constipation medication is administered concurrently with maralixibat or a pharmaceutically acceptable salt thereof. In some embodiments, the constipation medication is administered prior to the administration of maralixibat or a pharmaceutically acceptable salt thereof and is continued concurrently after the start of the administration of maralixibat or a pharmaceutically acceptable salt thereof. In some embodiments, a constipation medication is administered after the administration of maralixibat or a pharmaceutically acceptable salt thereof.

In some embodiments, maralixibat or a pharmaceutically acceptable salt thereof is administered prior to the administration of a constipation medication. In some embodiments, maralixibat or a pharmaceutically acceptable salt thereof is administered concurrently with a constipation medication. In some embodiments, maralixibat or a pharmaceutically acceptable salt thereof is administered after the administration of a constipation medication.

In some embodiments, maralixibat or a pharmaceutically acceptable salt thereof is administered after at least four weeks of the administration of a constipation medication.

The constipation medication may include any conventional constipation medication. Non-limiting examples of the constipation medication may include a laxative, a stool softener, a hygroscopic medication, a secretagogue, a prokinetic, a peripherally acting mu-opioid receptor agonist (PAMORA), lactulose, sorbitol, macrogol 3350, sodium phosphate, glycerol suppository, and combination thereof. Non-limiting examples of the laxative may include bisacodyl, glycerin, magnesium hydroxide, magnesium citrate, polyethylene glycol, psyllium, senna, sodium picosulfate, docusate, and combination thereof. In one embodiment, a non-limiting example of a laxative may include polyethylene glycol. In one particular embodiment, the laxative is polyethylene glycol 3350, also known as Miralax®. One non-limiting example of the hygroscopic medication is a fiber-based stool softener.

In various embodiments of the above methods of the invention, administration of maralixibat or maralixibat chloride results in a reduction in a symptom or a change in a disease-relevant laboratory measure of the constipation associated with cystic fibrosis (i.e., improvement in the patient's condition) that is maintained for about or for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years. In various embodiments, the reduction in the symptom or a change in a disease-relevant laboratory measure comprises an increase in BSS score, an improved ease of stooling, softer stool consistency, or a combination thereof. In various embodiments, the reduction in the symptom or a change in a disease-relevant laboratory measure comprises an increase in BSS score. In various embodiments, the reduction in the symptom or a change in a disease-relevant laboratory measure is determined relative to a baseline level. That is, the reduction in the symptom or a change in a disease-relevant laboratory measure is determined relative to a measurement of the symptom or a change in a disease-relevant laboratory measure prior to 1) changing a dose level of the ASBTI administered to the patient, 2) changing a dosing regimen followed for the patient, 3) commencing administration of the ASBTI, or 4) any other of various alterations made with the intention of reducing the symptom or a change in a disease-relevant laboratory measure in the patient. In various embodiments, the reduction in symptom or a change in a disease-relevant laboratory measure is a statistically significant reduction.

In various embodiments, the reduction in a symptom or a change in a disease-relevant laboratory measure of the constipation associated with cystic fibrosis is measured as a progressive decrease in the symptom or a change in a disease-relevant laboratory measure for about or for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years.

In various embodiments, the administration of the ASBTI results in an increase in the BSS score to about or by at least about 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold relative to baseline.

The BSS score scale is a scale that classifies stools on a scale of 1 to 7, ranging from the hardest to the softest. Experts consider grade 1, 2 and 3 to be uncharacteristically hard and indicative of constipation, while grades 6 and 7 are unusually loose and may indicate diarrhea. Healthcare professionals generally consider types 4 and 5 to be the most typical, indicating neither constipation nor diarrhea.

In some embodiments, the administration of the ASBTI results in an improvement in stool consistency. In various embodiments, the improvement in stool consistency is measured using a BSS score. In various embodiments, the administration of the ASBTI results in an increase in the BSS score on a scale of 1 to 10 of about or of at least about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, or 10. In various embodiments, the administration of the ASBTI results in an increase in the BSS score to 1.0 or higher. In various embodiments, the administration of the ASBTI results in an increase in the BSS score by about or by at least about 10%, 15%, 20%, 25%, 30%, 35%, 40%, 45%, 50%, 55%, 60%, 65%, 70%, 75%, 80%, 85%, 90%, 95%, or 100% relative to baseline. In various embodiments, an increased value relative to baseline of the BSS score is observed on 10%, 20%, 30%, 40%, 50%, 60%, 70%, 75%, 80%, 85%, 90%, 91%, 92%, 93%, 94%, 95%, 96%, 97%, 98%, 99%, or 100% of days.

In various embodiments, the administration of the ASBTI results in the reduction in the symptoms of constipation or a change in the BSS score by about 1 day, 2 days, 3 days, 4 days, 5 days, 6 days, 7 days, 8 days, 9 days, 10 days, 11 days, 12, days, 13 days, 14 days, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year.

In various embodiments, the BSS score is increased by at least about 1, or to at least about the score of 4 indicating normal stool consistency at about or by about 1 day, 2 days, 3 days, 4 days, 5 days, 6 days, 1 week, 2 weeks, 3 weeks, 4 weeks, 1 month, 5 weeks, 6 weeks, 7 weeks, 8 weeks, 2 months, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year.

### Dose Modulation

In various embodiments, the method includes modulating a dosage of maralixibat administered to the patient. In some embodiments, modulating a dosage of maralixibat may comprise escalating the dosage of maralixibat. In some embodiments, the modulation includes administering maralixibat at a first dose level to the patient for a first week. If the patient tolerates the first dose level, the dose level is increased to a second dose level for a second week. If the patient tolerates the second dose level, the dose level is increased to a third dose level for a third week. If the patient tolerates the third dose level, the dose level is increased to a fourth dose level for the remainder of the treatment study.

In some embodiments, the method comprises a dose escalation period. In one non-limiting embodiment, the dose escalation period comprises the following weekly steps: a) dose level 1:190 μg/kg maralixibat QD for 1 week; and b) dose level 2:380 μg/kg maralixibat QD for 2 weeks and optionally for the remaining duration of the administration. In another non-limiting embodiment, dose escalation steps may be delayed or reversed to improve tolerability.

### Pharmaceutical Compositions

In some embodiments, maralixibat is administered as a pharmaceutical composition comprising maralixibat or maralixibat chloride). Any composition described herein can be formulated for ileal, rectal and/or colonic delivery. In more specific embodiments, the composition is formulated for non-systemic or local delivery to the rectum and/or colon. It is to be understood that, as used herein, delivery to the colon includes delivery to sigmoid colon, transverse colon, and/or ascending colon. In still more specific embodiments, the composition is formulated for non-systemic or local delivery to the rectum and/or colon is administered rectally. In other specific embodiments, the composition is formulated for non-systemic or local delivery to the rectum and/or colon is administered orally.

Provided herein, in certain embodiments, is a pharmaceutical composition comprising a therapeutically effective amount of any compound described herein. In certain instances, the pharmaceutical composition comprises an ASBT inhibitor (e.g., maralixibat or maralixibat chloride).

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Mareel Decker, New York, N.Y., 1980; and Pharmaceutical Dosage Forms and Drug Delivery Systems, Seventh Ed. (Lippincott Williams & Wilkins 1999), all of which references are incorporated herein in their entirety for all purposes.

A pharmaceutical composition, as used herein, refers to a mixture of a compound described herein, with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the compounds described herein are either utilized singly or in combination with one or more additional therapeutic agents.

In some embodiments, one or more additional therapeutic agents may include a constipation medication. In some embodiments, the compounds described herein are either utilized singly or in combination with a constipation medication. The constipation medication may include any conventional constipation medication. Non-limiting examples of the constipation medication may include a laxative, a hygroscopic medication, a secretagogue, a prokinetic, a peripherally acting mu-opioid receptor agonist (PAMORA), lactulose, sorbitol, macrogol 3350, sodium phosphate, glycerol suppository, and combination thereof. Non-limiting examples of the laxative may include bisacodyl, glycerin, magnesium hydroxide, magnesium citrate, polyethylene glycol, psyllium, senna, sodium picosulfate, dosucsate and combination thereof. A non-limiting example of the hygroscopic medication is a fiber-based stool softener. In one embodiment, the laxative is polyethylene glycol. In one particular embodiment, the laxative is polyethylene glycol 3350, also known as Miralax®.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

In certain embodiments, a pharmaceutical compositions described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Mareel Decker, New York, N.Y., 1980; and Pharmaceutical Dosage Forms and Drug Delivery Systems, Seventh Ed. (Lippincott Williams & Wilkins 1999), all of which references are incorporated herein in their entirety for all purposes.

Moreover, in certain embodiments, the pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

### Routes of Administration, Dosage Forms, and Dosing Regimens

In some embodiments, the compositions described herein, and the compositions administered in the methods described herein are formulated to improve stool consistency. In certain embodiments, the compositions described herein are formulated for rectal or oral administration. In some embodiments, such formulations are administered rectally or orally, respectively. In some embodiments, the compositions described herein are combined with a device for local delivery of the compositions to the rectum and/or colon (sigmoid colon, transverse colon, or ascending colon). In certain embodiments, for rectal administration the composition described herein are formulated as enemas, rectal gels, rectal foams, rectal aerosols, suppositories, jelly suppositories, or retention enemas. In some embodiments, for oral administration the compositions described herein are formulated for oral administration and enteric delivery to the colon.

In certain embodiments, the compositions or methods described herein are non-systemic. In some embodiments, compositions described herein deliver the ASBTI to the distal ileum, colon, and/or rectum and not systemically (e.g., a substantial portion of the enteroendocrine peptide secretion enhancing agent is not systemically absorbed). In some embodiments, oral compositions described herein deliver the ASBTI to the distal ileum, colon, and/or rectum and not systemically (e.g., a substantial portion of the enteroendocrine peptide secretion enhancing agent is not systemically absorbed). In some embodiments, rectal compositions described herein deliver the ASBTI to the distal ileum, colon, and/or rectum and not systemically (e.g., a substantial portion of the enteroendocrine peptide secretion enhancing agent is not systemically absorbed). In certain embodiments, non-systemic compositions described herein deliver less than 90% w/w of the ASBTI systemically. In certain embodiments, non-systemic compositions described herein deliver less than 80% w/w of the ASBTI systemically. In certain embodiments, non-systemic compositions described herein deliver less than 70% w/w of the ASBTI systemically. In certain embodiments, non-systemic compositions described herein deliver less than 60% w/w of the ASBT1 systemically. In certain embodiments, non-systemic compositions described herein deliver less than 50% w/w of the ASBTI systemically. In certain embodiments, non-systemic compositions described herein deliver less than 40% w/w of the ASBTI systemically. In certain embodiments, non-systemic compositions described herein deliver less than 30% w/w of the ASBTI systemically. In certain embodiments, non-systemic compositions described herein deliver less than 25% w/w of the ASBTI systemically. In certain embodiments, non-systemic compositions described herein deliver less than 20% w/w of the ASBTI systemically. In certain embodiments, non-systemic compositions described herein deliver less than 15% w/w of the ASBTI systemically. In certain embodiments, non-systemic compositions described herein deliver less than 10% w/w of the ASBTI systemically. In certain embodiments, non-systemic compositions described herein deliver less than 5% w/w of the ASBTI systemically. In some embodiments, systemic absorption is determined in any suitable manner, including the total circulating amount, the amount cleared after administration, or the like.

In certain embodiments, the compositions and/or formulations described herein are administered at least once a day. In certain embodiments, the formulations containing the ASBTI are administered at least twice a day, while in other embodiments the formulations containing the ASBTI are administered at least three times a day. In certain embodiments, the formulations containing the ASBTI are administered up to five times a day. It is to be understood that in certain embodiments, the dosage regimen of composition containing the ASBTI described herein to is determined by considering various factors such as the patient's age, sex, and diet.

The concentration of the ASBTI administered in the formulations described herein ranges from about 1 mM to about 1 M. In certain embodiments the concentration of the ASBTI administered in the formulations described herein ranges from about 1 mM to about 750 mM. In certain embodiments, the concentration of the ASBTI administered in the formulations described herein ranges from about 1 mM to about 500 mM. In certain embodiments, the concentration of the ASBTI administered in the formulations described herein ranges from about 5 mM to about 500 mM. In certain embodiments, the concentration of the ASBTI administered in the formulations described herein ranges from about 10 mM to about 500 mM. In certain embodiments, the concentration of the administered in the formulations described herein ranges from about 25 mM to about 500 mM. In certain embodiments, the concentration of the ASBTI administered in the formulations described herein ranges from about 50 mM to about 500 mM. In certain embodiments, the concentration of the ASBTI administered in the formulations described herein ranges from about 100 mM to about 500 mM. In certain embodiments, the concentration of the ASBTI administered in the formulations described herein ranges from about 200 mM to about 500 mM.

In certain embodiments, by targeting the distal gastrointestinal tract (e.g., distal ileum, colon, and/or rectum), compositions and methods described herein provide efficacy (e.g., alleviating symptoms of constipation associated with cystic fibrosis) with a reduced dose of enteroendocrine peptide secretion enhancing agent (e.g., as compared to an oral dose that does not target the distal gastrointestinal tract).

### Liquid Dosage Forms

The pharmaceutical liquid dosage forms of the invention may be prepared according to techniques well-known in the art of pharmacy.

A solution refers to a liquid pharmaceutical formulation wherein the active ingredient is dissolved in the liquid. Pharmaceutical solutions of the invention include syrups and elixirs. A suspension refers to a liquid pharmaceutical formulation wherein the active ingredient is in a precipitate in the liquid.

In a liquid dosage form, it is desirable to have a particular pH and/or to be maintained within a specific pH range. In order to control the pH, a suitable buffer system can be used. In addition, the buffer system should have sufficient capacity to maintain the desired pH range. Examples of the buffer system useful in the present invention include but are not limited to, citrate buffers, phosphate buffers, or any other suitable buffer known in the art. Preferably the buffer system include sodium citrate, potassium citrate, sodium bicarbonate, potassium bicarbonate, sodium dihydrogen phosphate and potassium dihydrogen phosphate, etc. The concentration of the buffer system in the final suspension varies according to factors such as the strength of the buffer system and the pH/pH ranges required for the liquid dosage form. In one embodiment, the concentration is within the range of 0.005 to 0.5 w/v % in the final liquid dosage form.

The pharmaceutical composition comprising the liquid dosage form of the present invention can also include a suspending/stabilizing agent to prevent settling of the active material. Over time the settling could lead to caking of the active to the inside walls of the product pack, leading to difficulties with redispersion and accurate dispensing. Suitable stabilizing agents include but are not limited to, the polysaccharide stabilizers such as xanthan, guar and tragacanth gums as well as the cellulose derivatives HPMC (hydroxypropyl methylcellulose), methyl cellulose and Avicel RC-591 (microcrystalline cellulose/sodium carboxymethyl cellulose). In another embodiment, polyvinylpyrrolidone (PVP) can also be used as a stabilizing agent.

In addition to the aforementioned components, the ASBTI oral suspension form can also optionally contain other excipients commonly found in pharmaceutical compositions such as alternative solvents, taste-masking agents, antioxidants, fillers, acidifiers, enzyme inhibitors and other components as described in Handbook of Pharmaceutical Excipients, Rowe et al., Eds., 4^{th} Edition, Pharmaceutical Press (2003), which is hereby incorporated by reference in its entirety for all purposes.

Addition of an alternative solvent may help increase solubility of an active ingredient in the liquid dosage form, and consequently the absorption and bioavailability inside the body of a subject. Preferably the alternative solvents include methanol, ethanol or propylene glycol and the like.

In another aspect, the present invention provides a process for preparing the liquid dosage form. The process comprises steps of bringing maralixibat or its pharmaceutically acceptable salts thereof into mixture with the components including glycerol or syrup or the mixture thereof, a preservative, a buffer system and a suspending/stabilizing agent, etc., in a liquid medium. In general, the liquid dosage form is prepared by uniformly and intimately mixing these various components in the liquid medium. For example, the components such as glycerol or syrup or the mixture thereof, a preservative, a buffer system and a suspending/stabilizing agent, etc., can be dissolved in water to form the aqueous solution, then the active ingredient can be then dispersed in the aqueous solution to form a suspension.

In some embodiments, the liquid dosage form provided herein can be in a volume of between about 5 ml to about 50 ml. In some embodiments, the liquid dosage form provided herein can be in a volume of between about 5 ml to about 40 ml. In some embodiments, the liquid dosage form provided herein can be in a volume of between about 5 ml to about 30 ml. In some embodiments, the liquid dosage form provided herein can be in a volume of between about 5 ml to about 20 ml. In some embodiments, the liquid dosage form provided herein can be in a volume of between about 10 ml to about 30 ml. In some embodiments, the liquid dosage form provided herein can be in a volume of about 20 ml. In some embodiments, maralixibat can be in an amount ranging from about 0.001% to about 90% of the total volume. In some embodiments, maralixibat can be in an amount ranging from about 0.01% to about 80% of the total volume. In some embodiments, maralixibat can be in an amount ranging from about 0.1% to about 70% of the total volume. In some embodiments, maralixibat can be in an amount ranging from about 1% to about 60% of the total volume. In some embodiments, maralixibat can be in an amount ranging from about 1% to about 50% of the total volume. In some embodiments, maralixibat can be in an amount ranging from about 1% to about 40% of the total volume. In some embodiments, maralixibat can be in an amount ranging from about 1% to about 30% of the total volume. In some embodiments, maralixibat can be in an amount ranging from about 1% to about 20% of the total volume. In some embodiments, maralixibat can be in an amount ranging from about 1% to about 10% of the total volume. In some embodiments, maralixibat can be in an amount ranging from about 5% to about 70% of the total volume. In some embodiments, maralixibat can be in an amount ranging from about 5% to about 60% of the total volume. In some embodiments, maralixibat can be in an amount ranging from about 5% to about 50% of the total volume. In some embodiments, maralixibat can be in an amount ranging from about 5% to about 40% of the total volume. In some embodiments, maralixibat can be in an amount ranging from about 5% to about 30% of the total volume. In some embodiments, maralixibat can be in an amount ranging from about 5% to about 20% of the total volume. In some embodiments, maralixibat can be in an amount ranging from about 5% to about 10% of the total volume. In some embodiments, maralixibat can be in an amount ranging from about 10% to about 50% of the total volume. In some embodiments, maralixibat can be in an amount ranging from about 10% to about 40% of the total volume. In some embodiments, maralixibat can be in an amount ranging from about 10% to about 30% of the total volume. In some embodiments, maralixibat can be in an amount ranging from about 10% to about 20% of the total volume.

In one embodiment, the resulted liquid dosage form can be in a liquid volume of 10 ml to 30 ml, preferably 20 ml, and the active ingredient can be in an amount ranging from about 0.001 mg/ml to about 25 mg/ml, or from about 0.025 mg/ml to about 8 mg/ml, or from about 0.1 mg/ml to about 4 mg/ml, or about 0.25 mg/ml, or about 0.5 mg/ml, or about 1 mg/ml, or about 2 mg/ml, or about 4 mg/ml, or about 5 mg/ml, or about 8 mg/ml, or about 10 mg/ml, or about 12 mg/ml, or about 14 mg/ml or about 16 mg/ml, or about 18 mg/ml, or about 20 mg/ml, or about 25 mg/ml. In one embodiment, the active ingredient is maralixibat present in an amount of 9.5 mg/ml. In one embodiment, the active ingredient is maralixibat chloride present in an amount of 10 mg/ml.

### Oral Solution

In some embodiments, the pharmaceutical composition is formulated as an oral solution comprising maralixibat chloride, a preservative, an antioxidant, a flavoring agent, a sweetener, and water.

### Preservative

In certain embodiments, the compositions of the present invention comprise a preservative. In certain embodiments, the preservative is an antimicrobial preservative.

In certain embodiments, the antimicrobial preservative is selected from the group consisting of propylene glycol, ethyl alcohol, glycerin, benzalkonium chloride, benzethonium chloride, benzoic acid, benzyl alcohol, butylparaben, cetrimide (cetyltrimethylammonium bromide), cetrimonium bromide, cetylpyridinium chloride, chlorhexidine, chlorobutanol, chlorocresol, cresol, ethylparaben, methylparaben, phenol, phenoxyethanol, phenylethyl alcohol, phenylmercuric acetate, phenylmercuric borate, phenylmercuric nitrate, propylparaben, sodium benzoate, sodium dehydroacetate, sodium propionate, sorbic acid, potassium sorbate, thimerosal, thymol, and combinations thereof.

In certain embodiments, the preservative is propylene glycol.

In certain embodiments, the preservative is present in an amount of at least about 10% w/w of the composition. In certain embodiments, the preservative is present in an amount of at least about 20% w/w of the composition. In certain embodiments, the preservative is present in an amount of at least about 25% w/w of the composition. In certain embodiments, the preservative is present in an amount of at least about 30% w/w of the composition.

In certain embodiments, the preservative is present in an amount of from about 30% to about 40% of the composition.

In certain embodiments, the preservative is present in an amount of from about 32% to about 37% of the composition. In certain embodiments, the preservative is present in an amount of from about 33% to about 36% of the composition.

In certain embodiments, the preservative is present in an amount of about 33% of the composition. In certain embodiments, the preservative is present in an amount of about 34% of the composition. In certain embodiments, the preservative is present in an amount of about 35% of the composition.

### Antioxidant

In certain embodiments, the compositions of the present invention comprise an antioxidant. In certain embodiments, the antioxidant is selected from the group consisting of an aminocarboxylic acid, an aminopolycarboxylic acid, ascorbic acid, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, and combinations thereof.

In certain embodiments, the antioxidant is an aminopolycarboxylic acid selected from EDTA (ethylenediaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy) ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid).

In certain embodiments, the antioxidant is EDTA.

In certain embodiments, the antioxidant is present in an amount of about 0.001% to about 1% w/w of the composition. In certain embodiments, the antioxidant is present in an amount of about 0.005% to about 0.75% w/w of the composition. In certain embodiments, the antioxidant is present in an amount of about 0.01% to about 0.5% w/w of the composition. In certain embodiments, the antioxidant is present in an amount of about 0.05% to about 0.25% w/w of the composition. In certain embodiments, the antioxidant is present in an amount of about 0.075% to about 0.2% w/w of the composition. In certain embodiments, the antioxidant is present in an amount of about 0.1% w/w of the composition.

In some embodiments, the pharmaceutical composition comprises from about 5 mg/mL to about 50 mg/mL of maralixibat chloride; from about 300 mg/mL to about 400 mg/mL of propylene glycol; about 1 mg/mL of disodium EDTA; a sweetener, a flavoring agent, or a combination thereof, and water.

In some embodiments, the pharmaceutical composition comprises from about 5 mg/mL to about 50 mg/mL of maralixibat chloride; from about 300 mg/mL to about 400 mg/mL of propylene glycol; about 1 mg/mL of disodium EDTA; about 10 mg/mL sucralose, about 5 mg/mL grape flavor, and water.

In some embodiments, the pharmaceutical composition comprises about 10 mg/mL of maralixibat chloride (9.5 mg/mL of maralixibat); about 360 mg/mL of propylene glycol; about 1 mg/mL of disodium EDTA; about 10 mg/mL sucralose, about 5 mg/mL grape flavor, and water.

### Pediatric Dosage Formulations and Compositions

Provided herein, in certain embodiments, is a pediatric dosage formulation or composition comprising a therapeutically effective amount of any compound described herein. In certain instances, the pharmaceutical composition comprises an ASBT inhibitor (e.g., maralixibat or maralixibat chloride).

In certain embodiments, suitable dosage forms for the pediatric dosage formulation or composition include, by way of non-limiting example, aqueous or non-aqueous oral dispersions, liquids, gels, syrups, elixirs, slurries, suspensions, solutions, controlled release formulations, fast melt formulations, effervescent formulations, lyophilized formulations, chewable tablets, gummy candy, orally disintegrating tablets, powders for reconstitution as suspension or solution, sprinkle oral powder or granules, dragees, delayed release formulations, extended release formulations, pulsatile release formulations, multiparticulate formulations, and mixed immediate release and controlled release formulations. In some embodiments, provided herein is a pharmaceutical composition wherein the pediatric dosage form is selected from a solution, syrup, suspension, elixir, powder for reconstitution as suspension or solution, dispersible/effervescent tablet, chewable tablet, gummy candy, lollipop, freezer pops, troches, oral thin strips, orally disintegrating tablet, orally disintegrating strip, sachet, and sprinkle oral powder or granules.

In another aspect, provide herein is a pharmaceutical composition wherein at least one excipient is a flavoring agent or a sweetener. In some embodiments, provided herein is a coating. In some embodiments, provided herein is a taste-masking technology selected from coating of drug particles with a taste-neutral polymer by spray-drying, wet granulation, fluidized bed, and microencapsulation; coating with molten waxes of a mixture of molten waxes and other pharmaceutical adjuvants; entrapment of drug particles by complexation, flocculation or coagulation of an aqueous polymeric dispersion; adsorption of drug particles on resin and inorganic supports; and solid dispersion wherein a drug and one or more taste neutral compounds are melted and cooled, or co-precipitated by a solvent evaporation. In some embodiments, provided herein is a delayed or sustained release formulation comprising drug particles or granules in a rate controlling polymer or matrix.

Suitable sweeteners include sucrose, glucose, fructose or intense sweeteners, i.e. agents with a high sweetening power when compared to sucrose (e.g. at least 10 times sweeter than sucrose). Suitable intense sweeteners comprise aspartame, saccharin, sodium or potassium or calcium saccharin, acesulfame potassium, sucralose, alitame, xylitol, cyclamate, neomate, neohesperidine dihydrochalcone or mixtures thereof, thaumatin, palatinit, stevioside, rebaudioside, Magnasweet®. The total concentration of the sweeteners may range from effectively zero to about 300 mg/ml based on the liquid composition upon reconstitution.

In order to increase the palatability of the liquid composition upon reconstitution with an aqueous medium, one or more taste-making agents may be added to the composition in order to mask the taste of the ASBT inhibitor. A taste-masking agent can be a sweetener, a flavoring agent or a combination thereof. The taste-masking agents typically provide up to about 0.1% or 5% by weight of the total pharmaceutical composition. In a preferred embodiment of the present invention, the composition contains both sweetener(s) and flavor(s).

A flavoring agent herein is a substance capable of enhancing taste or aroma of a composition. Suitable natural or synthetic flavoring agents can be selected from standard reference books, for example Fenaroli's Handbook of Flavor Ingredients, 3rd edition (1995). Non-limiting examples of flavoring agents and/or sweeteners useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. Flavoring agents can be used singly or in combinations of two or more. In some embodiments, the aqueous liquid dispersion comprises a sweetening agent or flavoring agent in a concentration ranging from about 0.001% to about 5.0% the volume of the aqueous dispersion. In one embodiment, the aqueous liquid dispersion comprises a sweetening agent or flavoring agent in a concentration ranging from about 0.001% to about 1.0% the volume of the aqueous dispersion. In another embodiment, the aqueous liquid dispersion comprises a sweetening agent or flavoring agent in a concentration ranging from about 0.005% to about 0.5% the volume of the aqueous dispersion. In yet another embodiment, the aqueous liquid dispersion comprises a sweetening agent or flavoring agent in a concentration ranging from about 0.01% to about 1.0% the volume of the aqueous dispersion. In yet another embodiment, the aqueous liquid dispersion comprises a sweetening agent or flavoring agent in a concentration ranging from about 0.01% to about 0.5% the volume of the aqueous dispersion.

In certain embodiments, a pediatric pharmaceutical composition described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” for pediatric pharmaceutical compositions includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), all of which references are incorporated herein by reference in their entirety for all purposes.

Moreover, in certain embodiments, the pediatric pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In certain aspects, the pediatric composition or formulation containing one or more compounds described herein is orally administered for local delivery of maralixibat, maralixibat chloride, or other compounds described herein to the colon and/or rectum. Unit dosage forms of such compositions include a pill, tablet or capsules formulated for enteric delivery to colon. In certain embodiments, such pills, tablets or capsule contain the compositions described herein entrapped or embedded in microspheres. In some embodiments, microspheres include, by way of non-limiting example, chitosan microcores HPMC capsules and cellulose acetate butyrate (CAB) microspheres. In certain embodiments, oral dosage forms are prepared using conventional methods known to those in the field of pharmaceutical formulation. For example, in certain embodiments, tablets are manufactured using standard tablet processing procedures and equipment. An exemplary method for forming tablets is by direct compression of a powdered, crystalline or granular composition containing the active agent(s), alone or in combination with one or more carriers, additives, or the like. In alternative embodiments, tablets are prepared using wet-granulation or dry-granulation processes. In some embodiments, tablets are molded rather than compressed, starting with a moist or otherwise tractable material.

In certain embodiments, tablets prepared for oral administration contain various excipients, including, by way of non-limiting example, binders, diluents, lubricants, disintegrants, fillers, stabilizers, surfactants, preservatives, coloring agents, flavoring agents and the like. In some embodiments, binders are used to impart cohesive qualities to a tablet, ensuring that the tablet remains intact after compression. Suitable binder materials include, by way of non-limiting example, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), Veegum, and combinations thereof. In certain embodiments, diluents are utilized to increase the bulk of the tablet so that a practical size tablet is provided. Suitable diluents include, by way of non-limiting example, dicalcium phosphate, calcium sulfate, lactose, cellulose, kaolin, mannitol, sodium chloride, dry starch, powdered sugar and combinations thereof. In certain embodiments, lubricants are used to facilitate tablet manufacture; examples of suitable lubricants include, by way of non-limiting example, vegetable oils such as peanut oil, cottonseed oil, sesame oil, olive oil, corn oil, and oil of theobroma, glycerin, magnesium stearate, calcium stearate, stearic acid and combinations thereof. In some embodiments, disintegrants are used to facilitate disintegration of the tablet, and include, by way of non-limiting example, starches, clays, celluloses, algins, gums, crosslinked polymers and combinations thereof. Fillers include, by way of non-limiting example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. In certain embodiments, stabilizers are used to inhibit or retard drug decomposition reactions that include, by way of example, oxidative reactions. In certain embodiments, surfactants are anionic, cationic, amphoteric or nonionic surface active agents.

In some embodiments, maralixibat, maralixibat chloride, or other compounds described herein are orally administered in association with a carrier suitable for delivery to the distal gastrointestinal tract (e.g., distal ileum, colon, and/or rectum).

In certain embodiments, a pediatric composition described herein comprises maralixibat, maralixibat chloride, or other compounds described herein in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™ matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO3H) and swell in basic pH of the intestine (e.g., pH of about 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent to the distal ileum. In some embodiments, a dosage form comprising an enteroendocrine peptide secretion enhancing agent is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pediatric pharmaceutical composition described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20^{th} Edition (2000), a film coating is provided around the formulation of the compound of Formula I. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

In further embodiments, a tablet or capsule comprising an ASBTI or other compounds described herein is film-coated for delivery to targeted sites within the gastrointestinal tract. Examples of enteric film coats include and are not limited to hydroxypropylmethylcellulose, polyvinyl pyrrolidone, hydroxypropyl cellulose, polyethylene glycol 3350, 4500, 8000, methyl cellulose, pseudo ethylcellulose, amylopectin and the like.

### Solid Dosage Forms for Pediatric Administration

Solid dosage forms for pediatric administration of the present invention can be manufactured by standard manufacturing techniques. Non-limiting examples of oral solid dosage forms for pediatric administration are described below.

### Effervescent Compositions

The effervescent compositions of the invention may be prepared according to techniques well-known in the art of pharmacy.

Effervescent formulations contain and effervescent couple of a base component and an acid component, which components reach in the presence of water to generate a gas. In some embodiments, the base component may comprise, for example, an alkali metal or alkaline earth metal carbonate, or bicarbonate. The acid component may comprise, for example, an aliphatic carboxylic acid or a salt thereof, such as citric acid. The base and acid components may each independently constitute, for example, 25% to 55% (w/w) of the effervescent composition. The ratio of acid component to base component may be within the range of 1:2 to 2:1.

The effervescent compositions of the invention may be formulated using additional pharmaceutically acceptable carriers or excipients as appropriate. For example, one or more taste masking agents may be used. Dyes may also be used, as pediatric patients often prefer colorful pharmaceutical combinations. The compositions may take the form of, for example, tablets, granules or powders, granules or powders presented in a sachet.

### Chewable Tablets

The chewable tablets of the invention may be prepared according to techniques well-known in the art of pharmacy.

Chewable tablets are tablets that are intended to disintegrate in the mouth under the action of chewing or sucking and where, in consequence, the active ingredient has greater opportunity to come into contact with the bitter-taste receptors on the tongue.

One method of overcoming this issue is to absorb the active ingredient onto a suitable substrate. This approach is known in the art and described for example in U.S. Pat. No. 4,647,459, which is incorporated herein by reference in its entirety for all purposes.

Another approach involves forming the active ingredient into an aggregate along with a pre-swelled substantially anhydrous hydrocolloid. The hydrocolloid absorbs saliva and acquires a slippery texture which enables it to lubricate the particles of aggregate and mask the taste of the active ingredient. This approach is known in the art and described for example in European patent application 0190826, which is incorporated herein by reference in its entirety for all purposes.

Another approach involves employing a water-insoluble hygroscopic excipient such as microcrystalline cellulose. This approach is known in the art and described for example in U.S. Pat. No. 5,275,823, which is incorporated herein by reference in its entirety for all purposes.

In addition to the above approaches, the chewable tablets of the present invention can also contain other standard tableting excipients such as a disintegrant and a taste-masking agent.

### Orodispersible Tablets

The orodispersible tablets of the invention may be prepared according to techniques well-known in the art of pharmacy.

In orodispersible tablets of the invention, the excipient mixtures are such as to provide it with a disintegration rate so that its disintegration in the buccal cavity occurs in an extremely short time and especially shorter than sixty seconds. In some embodiments, the excipient mixture is characterized by the fact that the active substance is in the form of coated or non-coated microcrystals of microgranules. In some embodiments, the orodispersible tablet comprises one or several disintegrating agents of the carboxymethylcellulose type or insoluble reticulated PVP type, one or several swelling agents which may comprise a carboxymethylcellulose, a starch, a modified starch, or a microcrystalline cellulose or optionally a direct compression sugar.

### Powders for Reconstitution

The powder for reconstitution pharmaceutical compositions of the invention may be prepared according to techniques well-known in the art of pharmacy.

In some embodiments, the powder for reconstitution compositions of the invention comprise an effective amount of at least one internal dehydrating agent. The internal dehydrating agent can enhance the stability of the powder. In some embodiments, the internal dehydrating agent is magnesium citrate or disodium carbonate. In some embodiments, the powder composition comprises a pharmaceutically acceptable diluents, such as sucrose, dextrose, mannitol, xylitol, or lactose.

Powder compositions of the inventions may be placed in sachets or bottles for contemporaneous dissolution or for short term storage in liquid form (e.g. 7 days).

### Gummy Candies

The gummy candies of the invention may be prepared according to techniques well-known in the art of pharmacy.

Traditional gummy candy is made from a gelatin base. Gelatin gives the candy its elasticity, the desired chewy consistency, and a longer shelf life. In some embodiments, the gummy candy pharmaceutical composition of the invention includes a binding agent, a sweetener, and an active ingredient.

In some embodiments, the binding agent is a pectin gel, gelatin, food starch, or any combination thereof.

In some embodiments, the gummy candy comprises sweeteners, a binding agent, natural and/or artificial flavors and colors and preservatives. In some embodiments, the gummy candy comprises glucose syrup, natural cane juice, gelatin, citric acid, lactic acid, natural colors, natural flavors, fractionated coconut oil, and carnauba wax.

An ASBT inhibitor (e.g., maralixibat) may be used in the preparation of medicaments for the prophylactic and/or therapeutic treatment of cholestasis or a cholestatic liver disease (e.g., PFIC). A method for treating any of the diseases or conditions described herein in an individual in need of such treatment, may involve administration of pharmaceutical compositions containing at least one ASBT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

### EXAMPLES

The following examples illustrate specific aspects of the instant description. The examples should not be construed as limiting, as the examples merely provide specific understanding and practice of the embodiments and their various aspects.

### Example 1: Clinical Study Design of Maralixibat in Patients with Cystic Fibrosis and Constipation

This study is a within-subjects clinical pilot study to examine the effect maralixibat has on constipation in children (1-18 years of age) with cystic fibrosis (CF).

### Objectives

Primary objective: The primary aim of the study is to assess improvement in stool consistency in children (1-18 years of age) with CF. Constipation is defined as Bristol Stool Scale of ≤3 for 1 week prior to enrollment while on a stable laxative regimen for at least 4 weeks. It is investigated whether the addition of marlaixibat to a conventional constipation medication regimen can result in softer stool consistency (Bristol scale rating>3 after study drug-maralixibat) in patients with CF and constipation (Bristol scale rating≤3 before study drug-maralixibat).

Secondary objective: The secondary aims include assessment for improved ease of defecation (as subjectively judged by patients & their parents) after adding maralixibat to their constipation regimen. It is investigated whether there is subjective improvement in ease of stooling with the addition of maralixibat to a conventional constipation medication regimen via subjective questionnaire. Ease of defecation and ease of stooling are terms that are used interchangeably.

### Clinical Study Design

A total of 20 patients with CF and chronic constipation (defined as Bristol stool scale of ≤3 for 1 week prior to enrollment while on stable medication regimen for at least 4 weeks) are recruited. Patients receive IBATi (maralixibat) for 3 weeks in addition to their stable conventional constipation medication regimen.

Questionnaires via REDCap survey are administered by the investigators pre and post intervention to assess changes in stool consistency, frequency, and ease of defecation after adding maralixibat to their constipation regimen.

### Interventions and Duration of Clinical Study

A conventional constipation medication regimen alone for chronic constipation (stool softener, stimulant laxatives, dietary changes, etc.) is compared to conventional constipation regimen and maralixibat. Clinical information on conventional therapy is obtained via interview and patient chart review.
Conventional constipation regimen: stable regimen for at least 4 weeks
Conventional constipation regimen and maralixibat: 3 weeks
Week 1—Conventional therapy+maralixibat 190 mcg/kg once daily orally
Week 2—Conventional therapy+maralixibat 380 mcg/kg once daily orally
Week 3—Conventional therapy+maralixibat 380 mcg/kg once daily orally
Concentration of maralixibat: 9.5 mg/mL

Patients may be eligible for dose-escalation depending on the dose-escalation safety assessments.

Should patients experience adverse effects (including diarrhea, abdominal pain, and vomiting, liver test abnormalities, fat-soluble vitamin deficiency, nausea, gastrointestinal bleeding, and bone fractures) at 380 mcg/kg/dose, maralixibat can be held until symptoms resolve and can be resumed at 190 mcg/kg/dose. Should 190 mcg/kg/dose still produce adverse effects, subsequent decreases in dose or exclusion from the study can be considered.

Interventions may include the current existing standard of care based on clinical needs. Changes to conventional therapy for chronic constipation may be prohibited during the study, unless clinically indicated.

### Sample Size and Population of Clinical Study

Target population: Children (1-18 years of age) with CF and chronic constipation (defined as Bristol stool scale of ≤3 for 1 week prior to enrollment while on stable medication regimen for at least 4 weeks).
Number of participants: 20

This is a within-subjects study design, so there is no randomization and all patients receive the study intervention. The period prior to initiation of maralixibat serves as the ‘baseline’ or ‘control’ of our primary outcomes.

### Inclusion Criteria for Selection and Enrollment of Participants of Clinical Study

Age between 1-18 years;
Proven diagnosis of CF (via genetic testing or sweat chloride testing);
Proven diagnosis of chronic constipation (Bristol stool scale rating of ≤3 while on stable conventional therapy regimen); and
On stable (no medication changes or dose adjustments) conventional constipation medication regimen for chronic constipation (stool softeners, stimulant laxatives, dietary interventions) for at least 4 weeks.

### Exclusion Criteria for Selection and Enrollment of Participants of Clinical Study

Uncontrolled fat-soluble vitamin deficiency (Vitamin A, E, D, or K);
Abnormal liver-function tests (LFT) within 6-months prior to consent
Changes to conventional constipation medication regimen<4 weeks prior to initiation of maralixibat;
Adequately treated chronic constipation on conventional regimen (Bristol stool scale rating of >3);
Allergy or sensitivity to the study drugs (such as maralixibat and/or conventional constipation medication) their ingredients; and/or
Inability or unwillingness of individual or legal guardian/representative to given written informed consent.

### Study Enrollment Procedures

Subjects are identified throughout chart review or clinical encounters by study personnel.
A screening log is maintained documenting patients approached, consented, and participated/not participated, with ineligibility reasons.
Consent, assent, and parental permission forms are verbally discussed (via interpreter when necessary).

### Study Outcomes

### Primary Outcome(s)

Stool consistency using the Bristol Stool Scale (BSS)
   Constipation defined as Grade 3 or lower on BSS assessment.
   Softened stool defined as Grade 4 or higher on BSS assessment.

### Secondary Outcome(s)

Subjective ‘ease of stooling’ based on pre- and post-intervention questionnaire.

### Various Evaluations During Clinical Study

### Screening

All participants who consent to the study undergo the screening period.

Screening involves the administration of a screening questionnaire, administered by the investigators at the time of routine clinic appointments. This may determine if all inclusion criteria are met and none of the exclusion criteria are met.

### Enrollment

Enrollment date is defined as the day patient has met all inclusion criteria, has not met any exclusion criteria, and has understood and signed the consent/assent/parent permission forms.

### Baseline Assessments

Medical history and current medications are reviewed verbally and via the medical record (recorded in REDCap datasheet) for initial assessment to provide baseline for comparison in the post-study period.

Visit 1 (Baseline enrollment visit: day 0, in-person): Review of medical history and current medications along with the collection of informed consent form, assent form, parental permission form, and/or screening form.

### Follow-Up Visits

The study team conducts phone encounters with families at the start of week 2 (day 8) and the at the start of week 3 (day 15) to ensure no question, concerns, or adverse events are present.

Visit 2 (start of week 2) and visit 3 (start of week 3): Review of adverse events, if any. At Visit 2, the investigator determines the causality of any AEs to determine dosing eligibility.

### Final Evaluation

Medical history and current medications are reviewed verbally and via the medical record (recorded in REDCap datasheet) for post-study assessment at the end of week 3. The family measures the volume of the study drug left at the end of the study period, which is compared to the volume dispensed to ensure appropriate adherence during this telehealth appointment. Adverse events and symptoms are reviewed verbally (recorded in REDCap datasheet).

Visit 4 (Post-intervention visit: day 21 or beyond): Review of medical history, current medications, adverse events, and drug weight (adherence check)

Post-study assessment or assessment of visit 4 can be via telehealth appointment.

Patients who discontinue the study for any reason have the same evaluation on day 21 as patient who completed the study as intended.

### Adherence Assessment

Adherence is assessed through volume comparison of any unused study drug at the end of the study period, defined as at least 80% of the treatment intervention volume by weight taken. The cup supplied with the medication is utilized to ascertain the final volume. In the absence of a provided cup, the study team collaborates with the patient to establish an appropriate method for determining the final volume via telehealth.

### Safety Assessments

Safety parameters consist of clinical monitoring for known adverse symptoms of maralixibat.

Safety parameters are assessed/evaluated at the final clinic visit or during contact initiated by study patients at any point during the study period. All safety parameters are recorded in REDCap datasheet. Relevant adverse effects are present in drug package insert (12).

Adverse events: An adverse event (AE) is generally defined as any unfavorable and unintended diagnosis, symptom, sign (including an abnormal laboratory finding), syndrome or disease which either occurs during the study, having been absent at baseline, or if present at baseline, appears to worsen. Adverse events are to be recording regardless of their relationship to the study intervention. Potential adverse events are listed below.
Severe abdominal pain—defined as requiring acute medical intervention based on clinical assessment by qualified healthcare personnel
Severe diarrhea—defined as Bristol stool 6-7, >3× daily
Severe vomiting—defined as unable to maintain hydration based on clinical assessment by qualified healthcare personnel
Anaphylaxis—defined by clinical assessment by qualified healthcare personnel
Severe GI bleeding—defined by clinical assessment by qualified healthcare personnel

Serious adverse events: A serious adverse event (SAE) is generally defined as any untoward medical occurrence that results in death, is life threatening, requires inpatient hospitalization or prolongation of existing hospitalization, results in persistent or significant disability/incapacity, or is a congenital anomaly.

All AEs and SAEs relate data are collected at the post intervention visit (day 21) via patient/family interview or via medical record review (solicited events) or via patients/family direct contact with the investigators (unsolicited events). Solicited AEs/SAEs are only be collected at the post-intervention visit (day 21). Unsolicited events may be collected at any time during the study period. Double capture is avoided by cross-referencing all AEs/SAEs for each enrolled patient.

All AEs/SAEs are recorded via a source document created by the investigators outlining the events, severity, and relatedness/expectedness to the study medication (maralixibat). All recorded SAEs are additionally be reported to the IRB via iSTAR within 24 hours of knowledge of the event. SAEs should be reported as soon as they are determined to meet the definition, even if complete information is not yet available. During and after the study, any significant new findings related to the study product that may (i) adversely affect the safety, well-being, or medical care of subjects, (ii) affect the willingness of subjects to continue participation in the study, or (iii) alter the IRB's continuing approval of the study should be notified.

If an adverse event is thought to have occurred, weekly verbal follow-up is initiated by the investigators to follow adverse events to ensure resolution. Duration of the follow-up may include the duration of the study period (21 days)+14 days after completion of the study period (regardless of completion of the study intervention).

The investigator determines the causality of all AEs to the study drug using medical judgement and considering all relevant factors to the underlying study indication.

### Liver Monitoring

Substantial changes in vitamin levels may occur over prolonged exposure to the study medication affecting their absorption or metabolism. Liver function testing and fat-soluble vitamin levels are used as baseline but are not warranted beyond routine testing.

### Dose-Escalation Safety Assessments

For dose-escalation safety assessments, adverse events with attribution of unlikely or unrelated to study drug, the patient is eligible for dose escalation as determined by the Investigator(s). Patients who have experienced an AE with attribution of possibly, probably, or definitely related to study drug may be eligible for dose escalation on a case-by-case basis or may not be eligible for dose escalation as determined by the Investigator(s).

### Data Analyses

Demographic and subjective ‘ease of stooling’ information is descriptive. Comparison of pre- and post-intervention stool consistency is descriptive and is analyzed by 2-tailed T-test to assess for significant differences.

### Example 2: Maralixibat in Patients with Cystic Fibrosis and Constipation, A within-Subjects Feasibility Study

Background: Chronic constipation is common in children with cystic fibrosis (CF) with symptoms due to impaired chloride channel function and reduces gastrointestinal (GI) secretions. Current therapies require large-volume intake or abdominal cramping, posing challenges in pediatrics. Maralixibat, a non-systemic ileal bile acid transport inhibitor (IBATi) acts by interrupting bile acid reabsorption in the ileum, thus interrupting the normal enterohepatic circulation. This interruption results in a larger volume of bile acids reaching the colon. Bile acids accelerate transit and increase mucosal secretion through non chloride ionic channels. Adult studies in IBS-C, CIC, and functional constipation demonstrate improved stool frequency and reduced transit time with drugs like Maralixibat, such as Elobixibat. However, no pediatric studies have evaluated IBATi in CF-related constipation.
Primary aim: feasibility (screening, enrollment, retention, and data completeness). Efficacy endpoint: improvement of >1 Bristol point or a score>4. Secondary endpoint: improved ease of defecation.
Methods: A within-subjects feasibility pilot study was conducted to evaluate off-label Maralixibat use in pediatric patients with CF and chronic constipation, presenting to a tertiary pediatric center's specialty outpatient multidisciplinary clinic. Children, ages 1-18 years, with confirmed CF and chronic constipation on Bristol Stool Scale≤3 for ≥1 week on a stable regimen≥4 weeks, were recruited. Participants received three weeks of Maralixibat at package-insert dosing. Patients are recommended to maintain a stable laxative regimen if on established treatment. REDCap questionnaires administered pre- and post-intervention assessing stool consistency, frequency, and ease of defecation.
Results: 162 patients were screened; 17 (9.5%) met inclusion criteria. Of these, 7 consented (41.2%) and 4 completed the study (57.1% retention). Clinic-based screening was efficient given standardized documentation of Bristol stool scores and GI symptoms. REDCap data capture met targets with high survey completion among retained participants. Three participants withdrew due to, time constraint at clinic visit, intolerance to medication palatability, adverse event with abdominal pain post medication ingestion, later noted to be in the setting of concurrent fecal impaction on imaging at the emergency room.
Conclusion: A within-subjects Maralixibat trial in children with CF and constipation is feasible, with efficient screening and reliable data collection. However, enrollment and retention can be improved by engaging patients before their clinic visits and screening for symptoms of fecal impaction. Future trials should reduce participant burden to evaluate Maralixibat's therapeutic potential in pediatric CF constipation. Exploring alternative formulations, such as compounded medications or tablet versions, may also enhance adherence.

### REFERENCES

1. Prevalence of constipation in cystic fibrosis patients: a systematic review of observational studies. J Pediatr (Rio J). 2020 November-December; 96 (6): 686-692.
2. Livmarli (maralixibat) oral solution. Package Insert. Mirum Pharmaceuticals, Inc; 2021.
3. Farrugia A, Arasaradnam R, Bile acid diarrhoea: pathophysiology, diagnosis and management, Frontline Gastroenterology 2021; 12:500-507.
4. Chadwick V S, Gaginella T S, Carlson G L, et al., Effect of molecular structure on bile acid-induced alterations in absorptive function, permeability, and morphology in the perfused rabbit colon. J Lab Clin Med 1979; 94:661-74.pmid: http://www.ncbi.nlm.nih.gov/pubmed/501195
5. Mekhjian H S, Phillips S F, Hofmann A F. Colonic secretion of water and electrolytes induced by bile acids: perfusion studies in man. Journal of Clinical Investigation 1971; 50:1569 77.doi: 10.1172/JCI106644
6. Bampton P A, Dinning P G, Kennedy M L, et al., The proximal colonic motor response to rectal mechanical and chemical stimulation. Am J Physiol Gastrointest Liver Physiol 2002; 282: G443-9.
7. Johnston I M, Nolan J D, Pattni S S, et al., Characterizing factors associated with differences in FGF19 blood levels and synthesis in patients with primary bile acid diarrhea. Am J Gastroenterol 2016; 111:423 32.doi: 10.1038/ajg.2015.424 pmid: http://www.ncbi.nlm.nih.gov/pubmed/26856750
8. McJunkin B, Fromm H, Sarva R P, et al., Factors in the mechanism of diarrhea in bile acid malabsorption: fecal pH—a key determinant. Gastroenterology 1981; 80:1454 64.doi: 10.1016/0016-5085 (81) 902572 pmid: http://www.ncbi.nlm.nih.gov/pubmed/7227771
9. Simren M., Bajor A., Gillberg P. G., Rudling M., Abrahamsson H. (2011). Randomised clinical trial: the ileal bile acid transporter inhibitor A3309 vs. placebo in patients with chronic idiopathic constipation—a double-blind study. Aliment. Pharmacol. Ther. 34 41-50. 10.1111/j.1365-2036.2011.04675
10. Chey W. D., Camilleri M., Chang L., Rikner L., Graffner H. (2011). A randomized placebo-controlled phase IIb trial of a 3309, a bile acid transporter inhibitor, for chronic idiopathic constipation. Am. J. Gastroenterol. 1061803-1812. 10.1038/ajg.2011.162 Wong B. S., Camilleri M., McKinzie S., Burton D., Graffner H., Zinsmeister A. R. (2011). Effects of A 3309, an ileal bile acid transporter inhibitor, on colonic transit and symptoms in females with functional constipation. Am. J. Gastroenterol. 106 2154-2164. 10.1038/ajg.2011.285
11. Nakajima A., Seki M., Taniguchi S., Ohta A., Gillberg P. G., Mattsson J. P., et al. (2018b). Safety and efficacy of elobixibat for chronic constipation: results from a randomised, double-blind, placebo-controlled, phase 3 trial and an open-label, single-arm, phase 3 trial. Lancet Gastroenterol. Hepatol. 3 537-547. 10.1016/S2468-1253 (18) 30123-7
12. Livmarli (maralixibat) oral solution. Package Insert. Mirum Pharmaceuticals, Inc; 2021.
13. National Center for Biotechnology Information. “PubChem Compound Summary for CID 9831643, Maralixibat” PubChem, https://pubchem.ncbi.nlm.nih.gov/compound/Maralixibat
14. Maralixibat in Patients With Cystic Fibrosis and Constipation, A Within-Subjects Pilot Study; clinicaltrials.gov ID NCT06413368 https://clinicaltrials.gov/study/NCT06413368?term=Constipation%20in%20Cystic%20Fibrosis&rank=1

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A method for treating constipation associated with cystic fibrosis in a subject in need thereof comprising administering to the subject an effective amount of maralixibat, or a pharmaceutically acceptable salt thereof.

2. The method of claim 1, wherein the constipation is a chronic constipation.

3. The method of claim 1, wherein the constipation is constipation predominant irritable bowel syndrome (IBS-C), chronic idiopathic constipation (CIC), constipation due to distal intestinal obstruction syndrome (DIOS), and/or functional constipation.

4.-7. (canceled)

8. The method of claim 1, wherein the subject is administered a constipation medication prior to the administration of maralixibat, or a pharmaceutically acceptable salt thereof.

9. The method of claim 8, wherein the constipation medication is selected from a laxative, a stool softener, a hygroscopic medication, a secretagogue, a prokinetic, a peripherally acting mu-opioid receptor agonist (PAMORA), lactulose, sorbitol, macrogol 3350, sodium phosphate, glycerol suppository, and combination thereof.

10.-15. (canceled)

16. The method of claim 1, wherein the maralixibat or pharmaceutically acceptable salt thereof is administered in an amount from about 100 μg/kg/day to about 1200 μg/kg/day.

17. (canceled)

18. The method of claim 1, wherein the maralixibat or pharmaceutically acceptable salt thereof is administered in an amount of about 190 μg/kg/day to about 800 μg/kg/day.

19. The method of claim 1, wherein the maralixibat or pharmaceutically acceptable salt thereof is administered in an amount of about 190 μg/kg/day or about 380 μg/kg/day.

20.-24. (canceled)

25. The method of claim 1, wherein the pharmaceutically acceptable salt of maralixibat is maralixibat chloride.

26. The method of claim 1, wherein the subject is a pediatric subject.

27. (canceled)

28. The method of claim 1, wherein the maralixibat or pharmaceutically acceptable salt thereof is administered once daily (QD).

29. The method of claim 1, wherein the administration of maralixibat or pharmaceutically acceptable salt thereof increases a Bristol Stool Scale (BSS) score of the subject relative to baseline.

30.-34. (canceled)

35. The method of claim 1, wherein the maralixibat or pharmaceutically acceptable salt thereof is administered before a meal.

36. (canceled)

37. The method of claim 1, wherein the maralixibat is administered orally.

38. The method of claim 1, wherein the maralixibat is administered in the form of a pharmaceutical composition comprising maralixibat, or a pharmaceutically acceptable salt thereof, an antioxidant, and a preservative.

39. The method of claim 38, wherein the pharmaceutical composition is a liquid composition for oral administration.

40. The method of claim 39, wherein the liquid composition is an aqueous solution.

41. The method of claim 39, wherein the maralixibat is present in an amount of about 1 mg/mL to about 100 mg/mL of the composition.

42. (canceled)

43. (canceled)

44. The method of claim 39, wherein the maralixibat is present in an amount of about 9.5 mg/mL of the composition.

45. The method of claim 38, wherein the pharmaceutical composition is a solid composition for oral administration.
